(19) United States
(12) Patent Application Publication
(10) Pub. No.: US 2013/0065949 A1
(43) Pub. Date: Mar. 14, 2013

(54) MICRORNA MIRNA-31 AS A THERAPEUTIC APPROACH FOR THE TREATMENT OF CANCER

(71) Applicants: Martin M. Matzuk, Houston, TX (US); Chad Creighton, Houston, TX (US); Zhifeng Yu, Houston, TX (US); Michael Fountain, Houston, TX (US); Preethi Gunaratne, Houston, TX (US); Matthew A. Anderson, Houston, TX (US)

(72) Inventors: Martin M. Matzuk, Houston, TX (US); Chad Creighton, Houston, TX (US); Zhifeng Yu, Houston, TX (US); Michael Fountain, Houston, TX (US); Preethi Gunaratne, Houston, TX (US); Matthew A. Anderson, Houston, TX (US)

(73) Assignee: Baylor College of Medicine, Houston, TX (US)

(21) Appl. No.: 13/501,806

(22) PCT Filed: Oct. 14, 2010
(86) PCT No.: PCT/US2010/052661

§ 371 Date: Nov. 26, 2012

Related U.S. Application Data
(60) Provisional application No. 61/251,608, filed on Oct. 14, 2009.

(51) Int. Cl. A61K 31/7088; A61P 35/00

(52) U.S. Cl. 514/44 R

(57) ABSTRACT

The present invention generally concerns methods and compositions for treating and/or preventing cancer with microRNAs. In specific embodiments, the present invention encompasses a particular microRNA, microRNA-31, as useful in cancer therapy and/or prevention. Specific cancers may be treated, including at least ovarian, prostate, urothelial, osteosarcoma, and glioblastoma.

### CROSS-REFERENCE TO RELATED APPLICATIONS

The present invention claims priority to U.S. Provisional Patent Application Ser. No. 61/251,608, filed Oct. 14, 2009, which is incorporated by reference herein in its entirety.

### STATEMENT REGARDING FEDERALLY SPONSORED RESEARCH OR DEVELOPMENT

This invention was made with government support under Grant Nos. CA60651, T32GM008307; P30CA125123, and K12HD01426-02 awarded by the National Institutes of Health; and Grant No. 2U54HG003273-05 awarded by the National Human Genome Research Institute. The government has certain rights in the invention.

### TECHNICAL FIELD

The present invention generally concerns the fields of cell biology, molecular biology, and medicine, including cancer medicine. In specific cases the present invention concerns the field of cancer therapy using microRNA, for example.

### BACKGROUND OF THE INVENTION

An estimated 15,520 women died of ovarian cancer in 2008, making it the fifth leading cause of cancer-associated death in women (1-3). Many efforts have been made to characterize ovarian cancer at the molecular level. A number of different clinicopathologic features, including stage, grade, and histotypes, have each been associated with distinct patterns of gene expression. For example, oncogenes, such as MYC, and tumor suppressors, such as Rb and p53, are frequently misexpressed in ovarian cancers, particularly serous tumors, the most common ovarian cancer histotype (˜70% of ovarian cancers (4)). In addition, mutations in TP53, BRCA1, and BRCA2 are each more frequently observed in poorly differentiated, high-grade serous cancers, whereas mutations in KRAS and BRAF are more frequently observed in relatively well-differentiated, low-grade carcinomas (5-7).

MicroRNAs (miRNAs) are recently discovered small (˜22 nt), non-coding RNAs that play critical roles in regulating complex patterns of gene expression. Functionally, miRNAs bind to complementary sequences in the 3′ untranslated region (UTR) of target gene transcripts, leading to mRNA degradation and/or translational repression (8, 9). Thus, miRNAs add a whole new layer of complexity by which large numbers of genes and their biological processes can be broadly regulated. Dysregulation of miRNAs has been described in several human cancers (10-12), each cancer type having unique miRNA expression patterns that likely impact patterns of gene expression relevant to tumor pathogenesis (13). Microarray profiling studies have revealed altered miRNA expression in epithelial ovarian cancers (14-19). Recently, reduced let-7i expression has been linked to increased resistance to cis-platinum chemotherapy in ovarian cancer (20), and miR-335 and miR-130a were both found to be consistently underexpressed in chemotherapy-resistant ovarian cancer cells (21). However, functional roles for most of the miRNAs thought to be aberrantly expressed in ovarian cancer have yet to be defined.

Here, we generated comprehensive miRNA and gene expression profiles for ovarian cancer by comparing papillary serous ovarian cancer, the most common cause of ovarian cancer deaths in women, to established ovarian cancer cell lines and short-term primary cultures of normal ovarian surface epithelium (NOSE). To better understand whether and how differentially expressed miRNAs impact ovarian cancers, top candidate miRNAs were experimentally altered in cell culture systems. Our findings indicate that diminished levels of miR-31 in particular (attributed in part to genomic deletion at 9p21) are correlated with defects in the p53 pathway and play a key role in the initiation and progression of ovarian cancer as well as other cancers.

### BRIEF SUMMARY OF THE INVENTION

The present invention generally concerns methods and/or compositions for treating and/or preventing cancer in a mammal, including a human, dog, cat, horse, cow, goat, sheep, or pig, for example. The individual may be known to have cancer, may be suspected of having cancer, or may be at high risk for developing cancer (such as family history, age over 65, tobacco user, excessive exposure to sunlight or ionizing radiation or certain chemicals, exposure to certain viruses (such as HPV or Epstein Barr virus) or bacteria (such as Helicobacter pylori), poor diet, lack of physical activity, and/or being overweight).

In certain embodiments of the invention, cancer is treated and/or prevented with methods and/or compositions of the invention. The cancer may be of any kind, but in specific embodiments the cancer is of the ovary, lung, breast, prostate, pancreas, brain, blood, liver, colon, gall bladder, pituitary gland, spleen, esophagus, testis, cervix, kidney, salivary gland, anus, skin or thyroid. In a specific embodiment, the method further comprises administering an additional cancer therapy to the individual, such as surgery, radiation, chemotherapy, immunotherapy, or hormone therapy, for example.

Certain embodiments of the invention concern microRNA-31 as a tumor suppressor in cancer, such as ovarian cancer, including serous ovarian cancer. In specific embodiments, the invention treats and/or prevents one or more types of ovarian cancer. In particular, the invention treats epithelial tumors that arise from cells that line or cover the ovaries (including serous, endometrioid, mucinous, and clear cell types); germ cell tumors that arise from cells that are destined to form eggs within the ovaries; and sex cord-stromal cell tumors that begin in the connective cells that hold the ovaries together and produce female hormones (including granulosa stromal cell tumors, Sertoli- or Sertoli-Leydig cell tumors, lipid cell tumors, and/or gynandroblastomas).

In certain embodiments of the invention, individuals with a risk for developing ovarian cancer or that have ovarian cancer and had the following risk factors are treated: older women; those who have a first or second degree relative with the disease; those with mutations in specific genes (most notably BRCA1 and BRCA2, but also in genes for hereditary nonpolyposis colorectal cancer); infertile women or those with endometriosis; those who have never been pregnant; and those who use postmenopausal estrogen replacement therapy.

In particular embodiments, it is provided herein that miR31 is a novel anti-proliferative/tumor suppressor miRNA that functions in cancer cell lines that have mutations in the p53/CDKN2A pathways, for example. It is also provided herein that overexpression of miR31 down-regulated E2F2 and SKP2, while p21 and p27 were up-regulated.

In particular embodiments of the invention, miR31 is used as a combination with other cancer therapy, including chemotherapy, surgery, radiation, hormone therapy, and/or immunotherapy, for example. When appropriate, combination chemotherapy is employed including, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, everolimus, Olaparib, or any combination, analog and/or derivative variant of the foregoing The additional drug being chemotherapy may be delivered to the individual in any suitable manner, including intraperitoneally, intravenously, or orally, for example. In certain cases, the additional anti-cancer therapy works additively or synergistically with the compositions of the present invention.

In certain embodiments of the invention, the miRNA-31 is delivered as a miR31 minicircle, miR31 nanoparticles, miR31 lentivirus, or miR31 adenovirus. In particular aspects, the composition is delivered via viruses (e.g. adenovirus), the miR31 by itself, or it may be conjugated to other substances to get into the cell. In certain embodiments, one can deliver miR-31 along with other microRNAs. In at least certain cases, the miR-31 is delivered by any means in which siRNA can be delivered, for example. Certain examples include by liposomes, DOPC, gold nanoparticles, viruses, minivector, and so forth.

In some embodiments of the invention, the microRNA-31 is delivered in conjunction with other microRNAs, including, for example, miR-26a, miR-34a, let-7, mir-15a and mir-16-1 and so forth.

In one embodiment of the invention, there is a method of treating cancer in an individual, wherein the cancer is ovarian, urothelial, osteosarcoma, glioblastoma, or prostate, comprising the step of delivering a therapeutically effective amount of microRNA-31 to the individual.

In a certain embodiment of the invention, there is a method of treating cancer in an individual, wherein the cancer is deficient in at least one component of a p53, CDKN2A, and/or RB1 pathway, comprising the step of delivering a therapeutically effective amount of microRNA-31 to the individual.

In a specific embodiment, the cancer is deficient in p53 signaling.

In a specific embodiment, the microRNA-31 is delivered to the individual orally, intraperitoneally, or intravenously.

In a certain embodiment, the microRNA-31 is delivered in a viral vector, such as a lentiviral vector, adenoviral vector, adeno-associated, or retroviral vector.

In a particular embodiment, the microRNA-31 targets E2F2, TFDP2, MDM2, or STK40.

In certain methods of the invention, the method further comprises the step of delivering an additional anti-cancer therapy to the individual, such as surgery, chemotherapy, radiation, hormone therapy, immunotherapy, or a combination thereof.

### BRIEF DESCRIPTION OF THE DRAWINGS

FIG. 1 shows profiling of miRNA, mRNA, and DNA in human serous ovarian tumors and cell lines. (A) Heat map representation of miRNAs overexpressed (yellow) and underexpressed (blue) in both cell lines and tumors compared to NOSE. Rows, miRNAs; columns, profiled samples. (B) Heat map representation of gene transcripts overexpressed (yellow) and underexpressed (blue) in cancer versus normal. Rows, transcripts; columns, samples. (C) Numbers of predicted miRNA-mRNA functional pairs for each algorithm and intersection of algorithms based on anti-correlated expression in ovarian cancer. (D) Percentages of genes overexpressed or underexpressed (from part B) that were located in a cytoband regions showing consistent copy number gain (orange) or loss (blue). (E) Frequency plot of DNA copy number gains or losses in a panel of 14 serous tumors. Locations of microRNAs from part A are indicated. (F) DNA loss or gain in regions flanking miR-31 in 178 serous ovarian tumors from the TCGA.

FIG. 2 demonstrates that modulation of miR-31 impacts predicted gene targets. A) Cells were transfected with miR-31 (which is normally underexpressed/deleted in serous ovarian cancers, see FIG. 1A) and profiled for gene expression. Genes represented in the profile dataset were ranked by fold change (overexpression/control). GSEA evaluated enrichment within the miR-31-expressing cells for predicted miR-31 targets, as determined by the given algorithm (Miranda, green; PicTar, blue; TargetScan, red). Vertical bars along the x-axis of the GSEA plot denote the positions, within the ranked list, of genes in the given set. Negative GSEA curve denotes anti-enrichment. (B) QPCR analysis showing relative quantity of miR-31 predicted targets (each normally underexpressed in cancer) after miR-31 overexpression in OVCAR-8 cells. For each gene, “mir-31 mimic” group (with overexpression of miR-31) is lower than either of the two control groups (p<0.05, two-sided t-test, each comparison). Bars indicate standard error. (C) Anti-enrichment of E2F2 transcriptional targets within miR-31 over-expressing cells, as determined by GSEA.

FIG. 3 shows gene expression signature of miR-31 overexpression is correlated with progression in advanced stage ovarian tumors. (A) The expression patterns of the miR-31 gene signature in a panel of 243 advanced stage human serous ovarian tumors from Tothill et al. Tumors are ordered by the “miR-31 signature t-score” which measures the similarity of the tumor profile with the miR-31-inducible profile. (B) Kaplan-Meier analysis comparing the differences in risk of disease relapse between tumors showing activation (red line, t-score>0 at P<0.05) of the miR-31 signature, tumors showing deactivation (blue line, t-score<0 and P<0.05) of the signature, and tumor showing intermediate patterns (yellow line). Log rank test evaluates whether there are significant differences between any of the three arms. Univariate Cox test evaluates the association of the miR-31 signature t-score with patient outcome, treating the coefficient as a continuous variable.

FIG. 4 provides overexpression of miR-31 inhibits cancer cell proliferation in p53 pathway-inactivated cell lines from ovarian cancer, prostate cancer, or osteosarcoma. A) Expression patterns of E2F2, CDKN2A (p16 gene), and well-known p53-inducible targets (e.g. p21 or CDKN1A) in ovarian cancer cell lines and NOSE controls. The p53 and p16 gene status for these cell lines as described in the literature is indicated. B) MTS assay for effect of miR-31 overexpression on proliferation of OVCAR8 cells. C) Caspase 3/7 activity assay for effect of miR-31 on caspase-mediated apoptosis. D-I) As for part B, but for cell lines SKOV3, OVCA433, HEY, OVSAYO, U2OS (osteosarcoma), and PC3 (prostate), respectively. For each time point in parts B-I delineated by asterisk (“*”), differences are significant with P<0.05 (two-sided t-test). Error bars reflect results from three independent cultures.

FIG. 5 illustrates an exemplary model for the synergistic interactions of miR31, p16, and p53 in cancer. Green lines with arrowheads denote stimulatory pathways, while red lines that end with a flat line are inhibitory pathways. In certain embodiments of the invention, miR-31 is a novel tumor suppressor that functions in conjunction with mutations in the p53/CDKN2A pathway to cause ovarian cancer.

FIG. 6 shows the proliferation inhibitory effect of miR31 in certain cancer cell lines.

FIG. 7 demonstrates that miR31 overexpression does not alter the proliferation of other ovarian cancer cell lines with wt p53/CDKN2A.

FIG. 8 shows that the proliferation inhibitory effect of miR31 is associated with defects in the p53 pathway.

FIG. 9 demonstrates analysis of some exemplary miR-31 targets.

FIG. 10 shows that SKP2 plays critical roles in the cell proliferation inhibitory function of miR31, in certain embodiments of the invention.

FIG. 11 illustrates the additive effect of miR31 and everolimus on OVCAR8 cells.

FIG. 12 demonstrates the additive effect of miR31 and Olaparib on OVCAR8 cells.

FIG. 13 shows that miR31 sensitized OVCAR8 to Olaparib.

FIG. 14 shows that adenoviral vector Ad5-CMV-tRFP-miR31 inhibited OVCAR8 cell proliferation.

FIG. 15 demonstrates that MiR31 adenovirus downregulated miR31 targets CEBPA, E2F2 and STK40.

FIG. 16 shows that miR31 adenovirus dose-dependently inhibited OVCAR8 cell proliferation.

Other and further objects, features, and advantages would be apparent and eventually more readily understood by reading the following specification and be reference to the accompanying drawings forming a part thereof, or any examples of the presently preferred embodiments of the invention given for the purpose of the disclosure.

### DETAILED DESCRIPTION OF THE INVENTION

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The skilled artisan recognizes that miRNAs are transcribed as hairpin precursors and are then sequentially processed by the RNase III enzymes, Drosha and Dicer, to yield double-stranded intermediates bearing 2 nt, 3′ overhanging ends. The duplexes are imperfectly paired and are subsequently generated into cytoplasmic protein-RNA complexes referred to as RNA-Induced Silencing Complexes (RISCs), which mediate RNA silencing. Each RISC comprises a single-stranded small RNA guide that is bound to a member of the Argonaute family of proteins. The miRNA and Argonaute protein act together to bind and silence respective target mRNAs. Perfectly complementary targets are efficiently silenced by the endonucleolytic cleavage activity of some Argonaute proteins, but the vast majority of predicted targets in animals are only partially paired and likely cannot be cleaved. Instead, they bind RISC using the “seed” of the miRNA, nucleotides 2-7, and are translationally repressed and/or degraded.

The terms “microRNA” or “miRNA” or “miR” are used interchangeably herein refer to endogenous RNA molecules, which act as gene silencers to regulate the expression of protein-coding genes at the post-transcriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

During miRNA maturation in animals, the primary transcript is first processed to a stem-loop precursor and then the stem-loop is processed to yield a mature miRNA of about 22 nucleotides. These molecules can direct the cleavage of mRNA or they can interfere with productive translation of the mRNA, either of which results in reduced protein accumulation and hence the miRNAs are able to modulate gene expression and related cellular activities. miRNAs are important in development and differentiation, and thus the altered expression of miRNAs could be used to alter development and differentiation during tissue engineering and other applications. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways. Mimetics of miRNAs include, artificial miRNAs, and siRNAs are inefficient and are not effective for many small RNA sequences.

The term “pri-miRNA” refers to a precursor microRNA molecule having a microRNA sequence in the context of microRNA flanking sequences. A precursor microRNA, also referred to as large RNA precursors, are composed of any type of nucleic acid based molecule capable of accommodating the microRNA flanking sequences and the microRNA sequence. Examples of precursor microRNAs and the individual components of the precursor (flanking sequences and microRNA sequence) are provided herein. The invention, however, is not limited to the examples provided. The invention is based, at least in part, on the discovery of an important component of precursor microRNAs, that is, the microRNA flanking sequences. The nucleotide sequence of the precursor and its components may vary widely. In one aspect a precursor microRNA molecule is an isolated nucleic acid; including microRNA flanking sequences and having a stem-loop structure with a microRNA sequence incorporated therein.

A precursor microRNA molecule may be processed in vivo or in vitro to produce a mature microRNA (miRNA). A precursor microRNA molecule is processed in a host cell by a ribonuclease enzyme or enzymes. One example of a ribonuclease enzyme which processes precursor microRNA molecules is the RNase II ribonuclease Dicer.

The term “pre-miRNA” refers to the intermediate miRNA species from the processing of a pre-miRNA to a mature miRNA. Pre-miRNAs are produced from the processing of a pri-miRNA in the nucleus into a pre-miRNA. Pre-miRNAs undergo additional processing in the cytoplasm to form mature miRNA. Pre-miRNAs are approximately 70 nucleotides long, but can be less than 70 nucleotides or more than 70 nucleotides.

MicroRNAs (miRNAs) regulate complex patterns of gene expression, and the relevance of altered miRNA expression to ovarian cancer remains to be elucidated. By comprehensively profiling expression of both miRNAs and mRNAs in serous ovarian cancers, we identified hundreds of potential miRNA-mRNA targeting associations underlying cancer versus normal differences. Functional overexpression of miR-31, the most underexpressed miRNA in serous ovarian cancer, repressed predicted miR-31 gene targets including cell cycle regulator E2F2. MiR-31 is located along with CDKN2A/ARF/p16 at 9p21.3, a genomic region commonly deleted at least in ovarian, urothelial, osteosarcoma, and glioblastoma. P16 promotes p53 activity, and E2F2 overexpression in p53 wild-type cells normally leads via p16 to an induction of p53-dependent apoptosis. In a number of serous cancer cell lines (i.e., OVCAR8, OVCA433, and SKOV3) with a dysfunctional p53 pathway, miR-31 overexpression inhibited cancer cell proliferation and induced apoptosis; however, in other lines (i.e., HEY and OVSAYO) with functional p53, miR-31 had no effect. Additionally, the osteosarcoma cell line U2OS and the prostate cancer cell line PC3 (p16- and p53-deficient, respectively) were also sensitive to miR-31. Furthermore, miR-31 overexpression induced a global gene expression pattern in OVCAR8 associated with better prognosis in tumors from patients with advanced stage serous ovarian cancer, potentially impacting many genes underlying disease progression. The findings reveal that loss of miR-31 is associated with defects in the p53 pathway and functions in serous ovarian cancer and other cancers, indicating that patients with cancers deficient in p53 signaling might benefit from therapeutic delivery of miR-31.

### I. Exemplary miRNA-31 Embodiments

In certain embodiments of the invention, miRNA-31 in its mature or non-mature form is utilized in the invention. The exemplary sequence is found in Accession No. MI0000089 of the miRBase database. In certain cases, the miR-31 stem-loop comprises GGAGAGGAGGCAAGAUGCUGGCAUAGCUGUUGAACUGGGAACCUGCUAUGCCAA CAUAUUGCCAUCUUUCC (SEQ ID NO:7). In specific embodiments, the mature sequence comprises AGGCAAGAUGCUGGCAUAGCU (SEQ ID NO:8) (bases 2-8 (the seed sequence) are most important, in certain embodiments). In specific cases, the corresponding Minor miR* sequence comprises UGCUAUGCCAACAUAUUGCCAU (SEQ ID NO:9).

In certain embodiments, polynucleotides comprising one of SEQ ID NO:7, SEQ ID NO:8 or SEQ ID NO:9 are delivered to an individual in need thereof. In certain embodiments, the polynucleotides comprise additional sequences at the 5′ and/or 3′ end of SEQ ID NO:7, SEQ ID NO:8 or SEQ ID NO:9, for example. The length of the polynucleotide may be of any suitable length so long as it is processable to the desired particular microRNA. In specific cases, the polynucleotide is no more than 150, 140, 130, 125, 120, 110, 100, 90, 80, 75, 70, 60, 50, 40, 30, 25, 24, 23, 22, 21, or 20 nucleotides in length. In certain aspects, the polynucleotide is at least 18, 19, 20, 21, 22, 23, 24, 25, 26, 27, 28, 29, 30, 35, 40, 45, 50, 55, 60, 65, 70, or 75 or more nucleotides in length. In certain embodiments, the polynucleotide has 1, 2, 3, 4, 5, 6, 7, 8, 9, 10, or more nucleotide differences compared to SEQ ID NO:7, SEQ ID NO:8, or SEQ ID NO:9. In particular cases, the present invention concerns polynucleotides that are at least 70%, 75%, 80%, 85%, 90%, 95%, 97%, or 99% identical to SEQ ID NO:7, SEQ ID NO:8, or SEQ ID NO:9.

The term “microRNA flanking sequence” as used herein refers to nucleotide sequences including microRNA processing elements. MicroRNA processing elements are the minimal nucleic acid sequences which contribute to the production of mature microRNA from precursor microRNA. Often these elements are located within a 40 nucleotide sequence that flanks a microRNA stem-loop structure. In some instances the microRNA processing elements are found within a stretch of nucleotide sequences of between 5 and 4,000 nucleotides in length that flank a microRNA stem-loop structure. Thus, in some embodiments the flanking sequences are 5-4,000 nucleotides in length. As a result, the length of the precursor molecule may be, in some instances at least about 150 nucleotides or 270 nucleotides in length. The total length of the precursor molecule, however, may be greater or less than these values. In other embodiments the minimal length of the microRNA flanking sequence is 10, 20, 30, 40, 50, 60, 70, 80, 90, 100, 150, 200 and any integer there between. In other embodiments the maximal length of the microRNA flanking sequence is 2, 000, 2,100, 2, 200, 2,300, 2,400, 2,500, 2,600, 2,700, 2,800, 2,900, 3, 000, 3,100, 3,200, 3,300, 3,400, 3,500, 3,600, 3,700, 3,800, 3,900 4,000 and any integer there between.

The microRNA flanking sequences may be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. The artificial microRNA flanking sequences may be flanking sequences found naturally in the context of other microRNA sequences. Alternatively they may be composed of minimal microRNA processing elements which are found within naturally occurring flanking sequences and inserted into other random nucleic acid sequences that do not naturally occur as flanking sequences or only partially occur as natural flanking sequences. The microRNA flanking sequences within the precursor microRNA molecule may flank one or both sides of the stem-loop structure encompassing the microRNA sequence. Thus, one end (i.e., 5′) of the stem-loop structure may be adjacent to a single flanking sequence and the other end (i.e., 3′) of the stem-loop structure may not be adjacent to a flanking sequence. Preferred structures have flanking sequences on both lo ends of the stem-loop structure. The flanking sequences may be directly adjacent to one or both ends of the stem-loop structure or may be connected to the stem-loop structure through a linker, additional nucleotides or other molecules.

### II. Delivery of MicroRNAs

The microRNAs of the present invention may be delivered in a variety of ways to an individual in need thereof. Certain embodiments include miR31 minicircle, miR31 nanoparticles, miR31 lentivirus, or miR31 adenovirus. In particular aspects, the composition is delivered via viruses (e.g. adenovirus, adeno-associated virus, retrovirus, or lentivirus), the miR31 by itself, or it may be conjugated to other substances to get into the cell. In certain embodiments, one can deliver miR-31 along with other microRNAs. In at least certain cases, the miR-31 is delivered by any means in which siRNA can be delivered, for example. Certain examples include by liposomes, DOPC, gold nanoparticles, viruses, minivector, and so forth. When delivered in viral vectors, miRNAs are continually transcribed, allowing sustained high level expression in target tissues.

The use of tissue-specific promoters restricts expression to particular cell types of interest, in certain embodiments of the invention. Exemplary tissue-specific promoters from ovary includes aromatase cytochrome P450 (Means et al., 1991), for example.

### III. Pharmaceutical Preparations

Pharmaceutical compositions of the present invention comprise an effective amount of one or more compositions of the invention dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one composition of the invention will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The compositions of the invention may be contained in different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The composition of the invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include a composition of the invention and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the composition of the invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes. In specific embodiments, the composition of the invention is administered to an individual in a liposome.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the compositions of the invention are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, compositions of the invention may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Combination Therapy

In some embodiments, the present invention is administered to an individual when the individual has been or is currently being treated with another cancer treatment or will be treated with another cancer treatment, or a combination thereof.

Thus, in some embodiments, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

It is contemplated that one or more compositions of the invention could be used similarly in conjunction with chemotherapeutic, radiotherapeutic, or immunotherapeutic intervention, in addition to other pro-apoptotic or cell cycle regulating agents. Alternatively, the composition(s) of the invention may precede or follow the other agent treatment by intervals ranging from minutes to weeks. In embodiments where the other agent and expression construct are applied separately to the cell, one would generally ensure that a significant period of time did not expire between the time of each delivery, such that the agent and composition of the invention would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed; the composition of the invention is “A” and the secondary agent, such as radio- or chemotherapy, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

A. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with a composition of the invention. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a secondary gene therapy in which a second therapeutic polynucleotide is administered before, after, or at the same time as the therapy with the composition(s) of the invention. Delivery of a vector encoding one of the following gene products will have an anti-hyperproliferative effect on target tissues. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl 2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl 2 protein now is recognized to be a member of a family of related proteins, which can be categorized as death agonists or death antagonists.

Subsequent to its discovery, it was shown that Bcl 2 acts to suppress cell death triggered by a variety of stimuli. Also, it now is apparent that there is a family of Bcl 2 cell death regulatory proteins which share in common structural and sequence homologies. These different family members have been shown to either possess similar functions to Bcl 2 (e.g., BclXL, BclW, BclS, Mcl-1, A1, Bfl-1) or counteract Bcl 2 function and promote cell death (e.g., Bax, Bak, Bik, Bim, Bid, Bad, Harakiri).

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### V. Kits of the Invention

Any of the compositions described herein may be comprised in a kit. The kits will thus comprise, in suitable container means, at least one composition encompassed in embodiments of the present invention.

The kits may comprise a suitably aliquoted composition of the invention. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the compositions in close confinement for commercial sale. Such containers may include injection or blow molded plastic containers into which the desired vials are retained, for example.

In certain embodiments of the invention, reagents and/or devices utilized in sample collecting are provided, such as a needle and vial. Other devices may be included, for example, a means to collect a biopsy sample, may be provided, such as a scalpel, needle, and so forth.

### EXAMPLES

The following examples are offered by way of example and are not intended to limit the scope of the invention in any manner.

### Example 1

### miRNA Expression Profiles of Serous Ovarian Cancer and Integration with mRNA and DNA Profiling Data

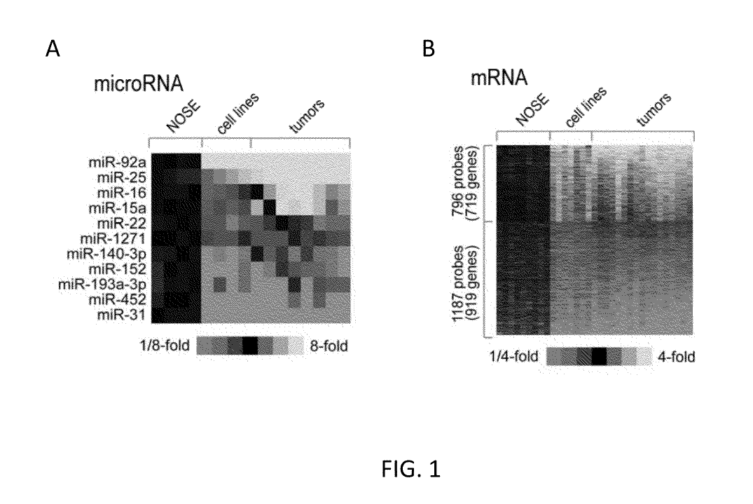

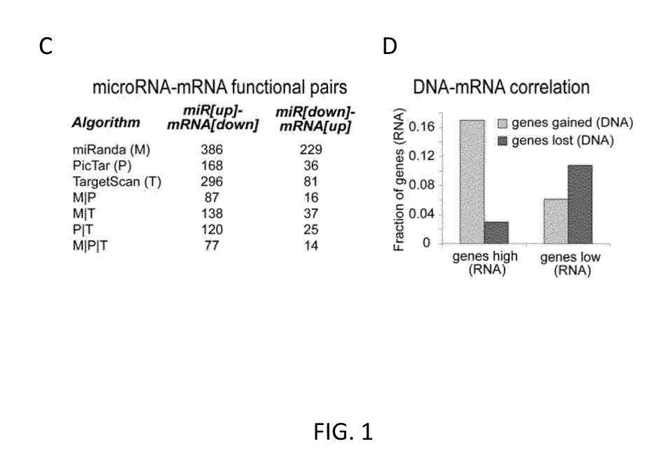

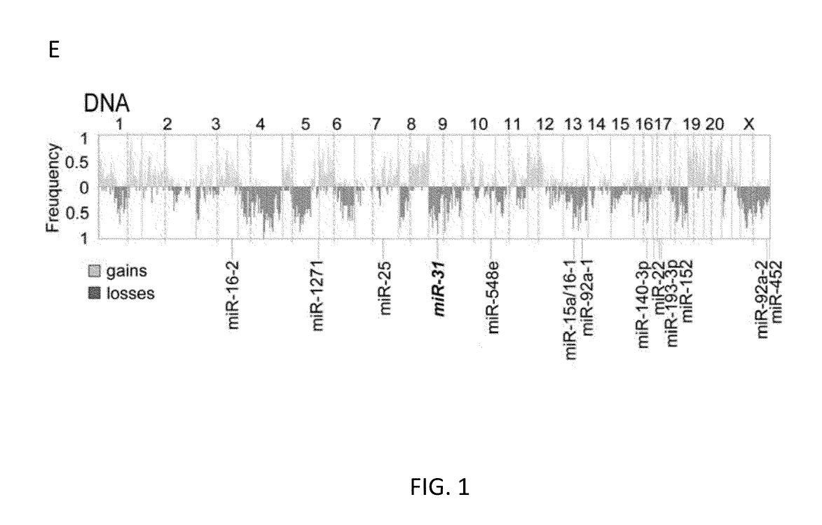

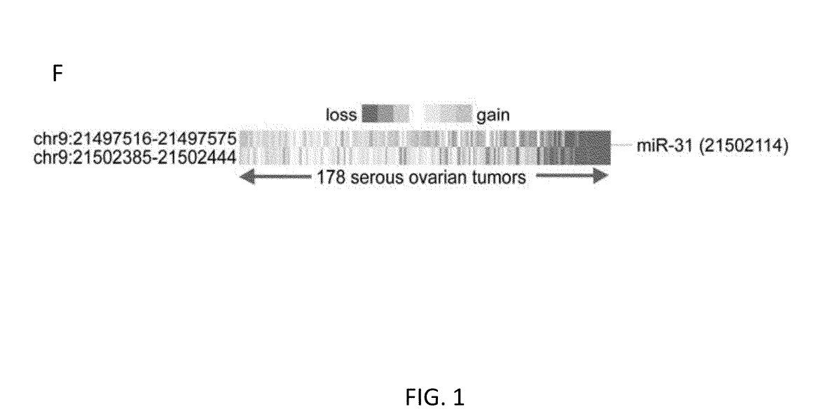

The inventors used deep sequencing to exhaustively identify the repertoire of ˜18-30 nucleotide small RNAs expressed in primary human serous ovarian cancers (n=8), cell lines established from serous ovarian cancers (i.e., SKOV3, HEY, OVCAR-5, OVCAR-8), and short-term primary cultures of human normal ovarian surface epithelium (NOSE, n=4). Approximately 50-70% of the (up to millions of) small RNA sequence reads for each sample mapped to known mature miRNAs in miRBase, with a total of 369 miRNAs detected in at least one sample. We found widespread differences between both cancer cell lines versus NOSE (47 miRNAs significant with p<0.01, chance expected from multiple testing=4) and tumors versus NOSE (42 miRNAs with p<0.01). The top 11 miRNAs over-expressed or under-expressed in both cancer cell lines and tumors (p<0.01, fold change>2, each comparison) are shown in FIG. 1A. We found good agreement between our set of serous ovarian cancer-associated miRNAs and the results from a recent study from Wyman et al. (19), which also used deep sequencing to profile miRNAs in ovarian cancer.

To help examine the potential impact of global shifts in miRNA expression on the genes expressed in ovarian cancer (by virtue of miRNA-mediated mRNA destabilization), we generated gene expression profiles of serous tumors (n=17, 8 of which were also profiled for miRNA), serous cancer cell lines (the four above, plus OVCAR-3, OVCA433, and Ov-90-B), and NOSE (n=9). The gene array platform represented 48,803 mRNA transcript probes, or ˜24K unique named genes. We found 796 mRNA transcript probes (719 genes) higher and 1187 probes (919 genes) lower in both cancer cell lines and tumors versus NOSE (P<0.01, fold change>1.5, each comparison) (FIG. 1B). To retrieve putative miRNA:mRNA functional pairs, we integrated our gene and miRNA expression profile results using the public target prediction databases TargetScan, miRanda, and PicTar. By our definition, a miRNA:mRNA functional pair consisted of a miRNA being predicted to interact with a given mRNA, where the two were also anti-correlated with each other in terms of expression in cancer versus normal. Both the “miRNA-down:mRNA-up” pairs (i.e., miRNA low in cancer, mRNA high in cancer) and the “miRNA-up:mRNA-down” pairs are determined, and the overall numbers in each category (for each algorithm or intersection of algorithms) are shown in FIG. 1C.

DNA copy number alterations in cancer have been established as having a major direct role in gene and miRNA transcription patterns (16, 22). To determine whether our gene expression results (given the basis of comparison) were consistent with this notion, we obtained a set of CGH profiles from 14 serous tumors from an independent study (23), from which we estimated which cytoband regions were gained or lost in at least 50% of the tumors. There was strong overlap between genes overexpressed in cancer and genes in regions of copy gain in cancer (with 17% of the 576 overexpressed genes represented in the CGH data showing gains, p<5E-5, one-sided Fisher's exact), as well as between genes under-expressed and in regions of loss (FIG. 1D). From a plot showing the frequency of DNA copy number gains and losses (FIG. 1E), a number of our deregulated miRNAs also appeared in regions of gain or loss, including miR-31 (underexpressed 35-fold, ranging from 12- to 460-fold, in our set of tumors and showing loss in >60% of tumors at 9p21.3), which was also found in an independent study to inhibit metastasis in breast cancer (24). We checked an additional CGH dataset of 178 advanced stage serous tumors from The Cancer Genome Atlas (TCGA, see the corresponding website) and found that the two CGH probes flanking miR-31 showed clear loss in about 25% of those tumors (FIG. 1F).

### Example 2

### Modulation of MIR-31 Impacts Predicted Gene Targets Including E2F2

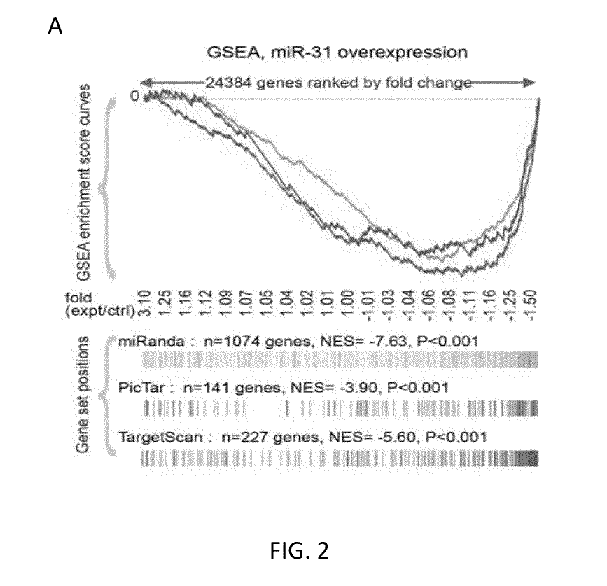

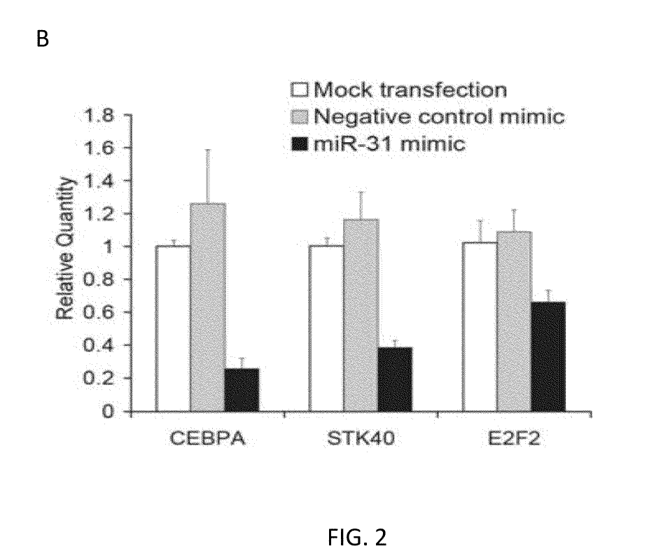

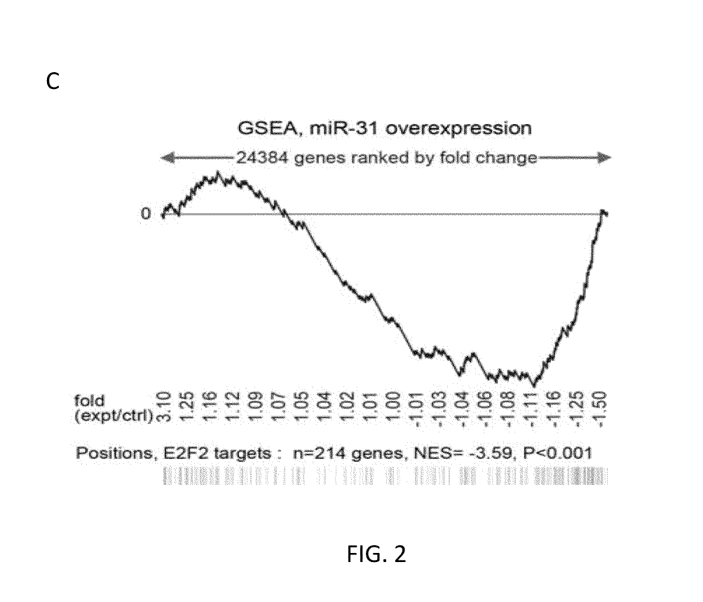

The bioinformatic analyses resulted in a large collection of hundreds of putative miRNA-mRNA interactions. Reversing the expression of candidate oncogenic or tumor suppressor miRNAs should conceivably cause a concomitant reversal of expression patterns for in silico predicted gene targets, including those targets aberrantly expressed in cancer. Using miRNA mimics in OVCAR-8 serous ovarian cancer cells, we overexpressed miR-31, which we had found to be both underexpressed and deleted in cancer, and therefore a candidate tumor suppressor. We then compared gene expression profiles between miR-31-transfected cells and cells transfected with a mimic control. From these data, we constructed a list of the profiled genes ordered according to higher expression in miR-31 over control (that is, the gene most induced would be at the top of this list, and the gene most repressed, at the bottom). We next used Gene Set Enrichment Analysis (GSEA) to capture, within this ordered list, the positions of predicted miR-31 target genes, separately considering the miRanda, PicTar, and TargetScan algorithms. By eye, it was apparent that predicted targets were in general repressed by miR-31 (FIG. 2A), a pattern found to be statistically significant by GSEA (which yielded negative Enrichment Score (ES) curves), regardless of the algorithm considered (p<0.001 for each).

Expression differences resulting from miR-31 overexpression were widespread, with 3922 genes (4802 probes) differing with p<0.01 (chance expected ˜488 probes). Of the TargetScan-predicted miR-31 gene targets repressed by miR-31 overexpression, we validated three—STK40, CEBPA, and E2F2—by QPCR (FIG. 2B), all three of which were also overexpressed in cancer versus normal and included in our putative miRNA:mRNA pairs. In addition to the predicted targets, many other genes appeared to be modulated by miR-31 (FIG. 2A), and other genes moved in the opposite direction from most targets. While any one prediction algorithm likely yields many false positives, it was also conceivable that many of the miRNA-modulated genes were indirect rather than direct targets (e.g., when a miRNA modulates a transcription factor). We were able to demonstrate this in the case of E2F2, a key regulator of cell cycle genes. We obtained a set of 214 E2F2 transcriptional targets derived using published datasets from models of Drosophila melanogaster (25, 26), in which the promoters of the targets were bound by E2F2, the targets were induced (>2-fold) by E2F2 overexpression, and human orthologs of these targets were represented in our data. GSEA demonstrated significant anti-enrichment of the E2F2 targets as a group within the miR-31 overexpression cells (FIG. 2C, p<0.001), where only 9 of the 214 genes were direct miR-31 targets by TargetScan.

### Example 3

### miR-31 Expression Induces a Gene Expression Signature Correlated with Better Outcome in Advanced Stage Serous Ovarian Tumors

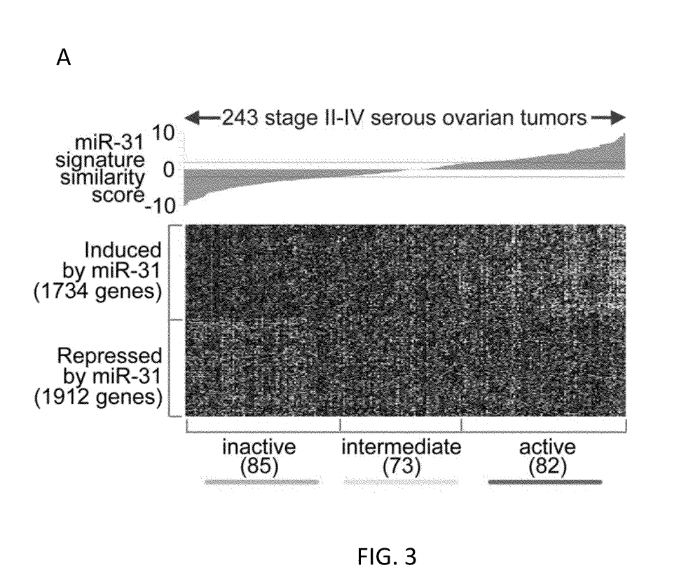

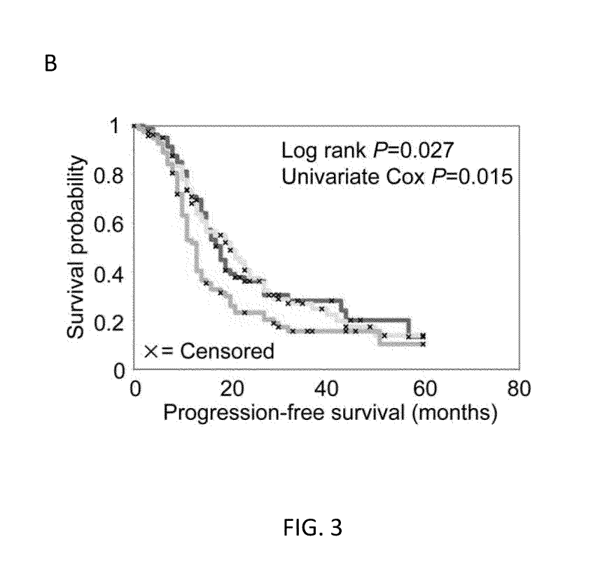

We obtained the publicly-available dataset from Tothill et al. (27) of gene expression profiles from 243 advanced stage (II-IV) serous tumors. In these tumors, we examined the expression patterns for the genes modulated (P<0.01) by miR-31 in vitro to determine whether the miR-31 signature is present in patients and whether its presence correlates with better clinical outcome, which would further indicate that miR-31 has potential tumor suppressive abilities. Using the 3646 miR-31 signature genes represented in the Tothill dataset, each Tothill serous ovarian tumor was assigned a miR-31 “t-score,” which gave a measure of how the tumor recapitulated the miR-31-induced patterns of over- and under-expression (FIG. 3A). Using this approach, we found that the level of enrichment of the tumors for the miR-31 signature was informative from a prognostic standpoint in the advanced stage serous tumors (FIG. 3B). Those patients with tumors clearly anti-correlated in expression patterns with the signature (t-score<0 at significance level P<0.05) had a significantly shorter median time to relapse event than did those patients with tumors either clearly correlated with the signature (t-score>0 at P<0.05) or with no strong patterns either way (P<0.03, Kaplan-Meier analysis, univariate Cox P<0.02 when treating the t-score as a continuous variable), suggesting that loss of miR-31 activity would aid in tumor progression.

### Example 4

### miR-31 Expression Inhibits Proliferation in Ovarian Cancer, Prostate Cancer, and Osteosarcoma

To assay the functional consequences of miR-31 overexpression in cancer, we used lentivirus to stably overexpress miR-31 in vitro. Because cancers of a particular histological subtype can be quite heterogeneous at the molecular level, we tested the effect of miR-31 in multiple cancer cell lines. We noted a number of connections between miR-31 and the p53 pathway. In particular, the gene encoding the tumor suppressor, p16, also called cyclin dependent kinase inhibitor 2A (CDKN2A, or ARF) is located along with miR-31 at human chromosome 9p21.3, a region commonly deleted in cancers (28); p16 functions as a tumor suppressor sequestering MDM2, the E3 ubiquitin ligase that directs p53 for degradation (29, 30). Furthermore, miR-31 suppresses E2F2, and the p53-dependent apoptotic program is often triggered by elevated levels and activity of E2F1 or E2F2 (whereby E2Fs upregulate p16, which blocks MDM2, leading to p53 accumulation)(31, 32). It would follow that inactivation of the p53 pathway (e.g. through p53 mutation or CDKN2A deletion) provides a growth advantage in those cancers with low miR-31 and (correspondingly) high E2F2 levels.

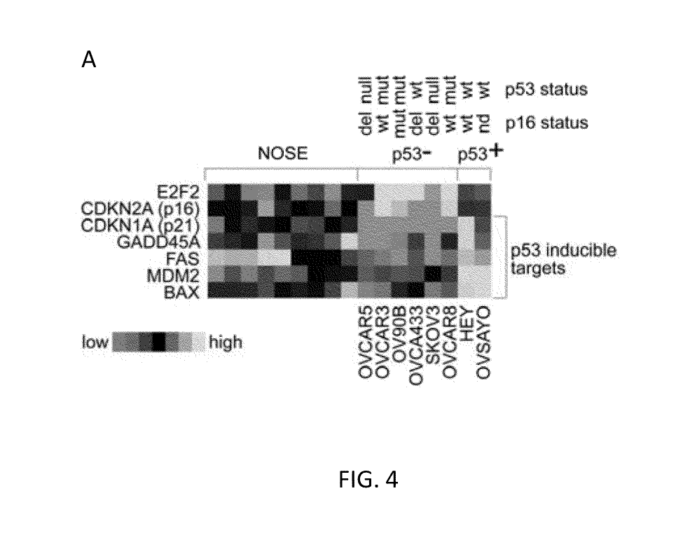

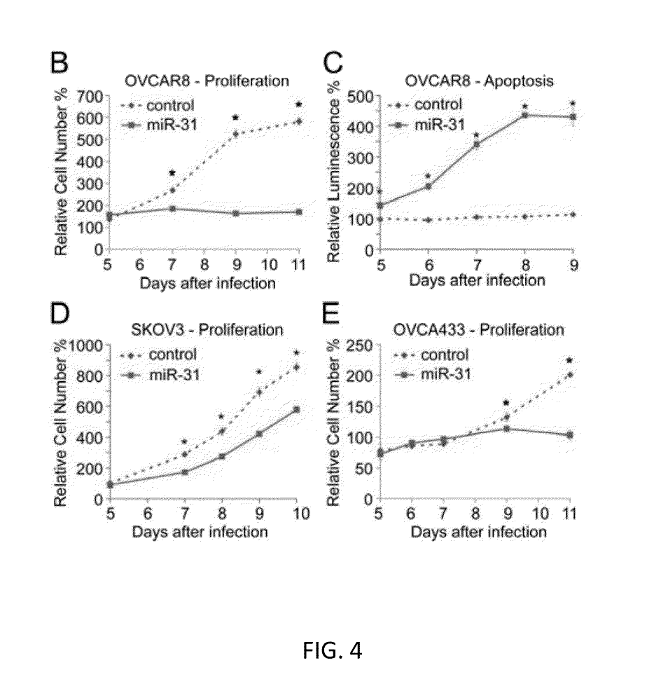

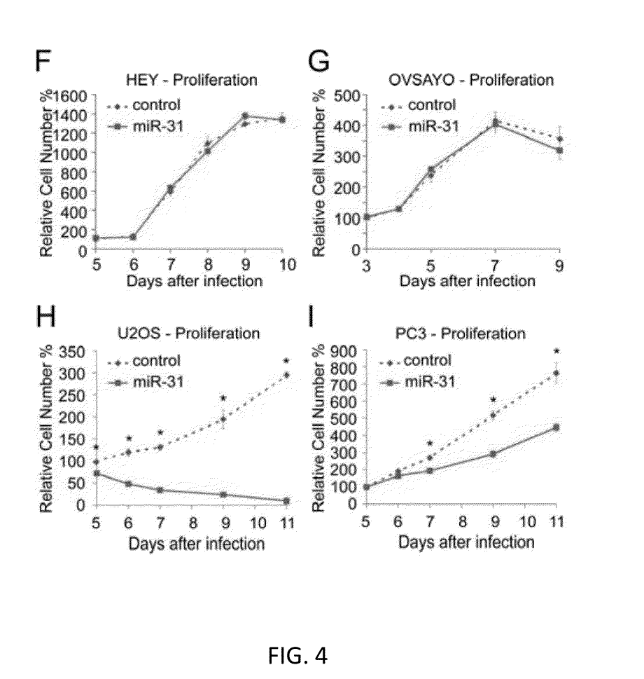

Using the gene expression data of the ovarian cancer cell lines and normal controls, we examined expression patterns of E2F2, CDKN2A (p16), and well-established p53-inducible transcriptional targets (e.g., p21)(FIG. 4A). On the basis of both the mRNA patterns and the p53 and p16 gene statuses of these cell lines as documented by the literature (33-40), our cell lines could be separated into those with a nonfunctional p53 pathway and those with a functional p53 pathway. Infection of the serous ovarian cancer line, OVCAR-8 (p53-deficient), with a lentivirus encoding miR-31 slowed proliferation (FIG. 4B) and caused the cells to undergo caspase-mediated apoptosis (FIG. 4C) compared to a control lentivirus expressing a non-targeted miRNA sequence. We went on to overexpress miR-31 in two other serous ovarian cancer cell lines with nonfunctional p53 pathways, SKOV3 and OVCA433, where a significant inhibition of proliferation was similarly observed (FIGS. 4D and 4E, respectively). However, in serous ovarian cancer cell line, HEY, and a clear cell ovarian cancer cell line, OVSAYO, two cell lines with wild-type p53 and p16 loci (in which, interestingly, E2F2 did not appear over-expressed, FIG. 4A), miR-31 had no effect on proliferation (FIGS. 4F and 4G, respectively). Importantly, we found the anti-proliferative effects of miR-31 were not unique to p53-deficient serous ovarian cancers, since both the osteosarcoma cell line U2OS (p53 WT; p16 mutant(41)) and the prostate cancer cell line PC3 (p53 null; p16 null(42)) were also sensitive to miR-31 (FIGS. 4H and 4I, respectively).

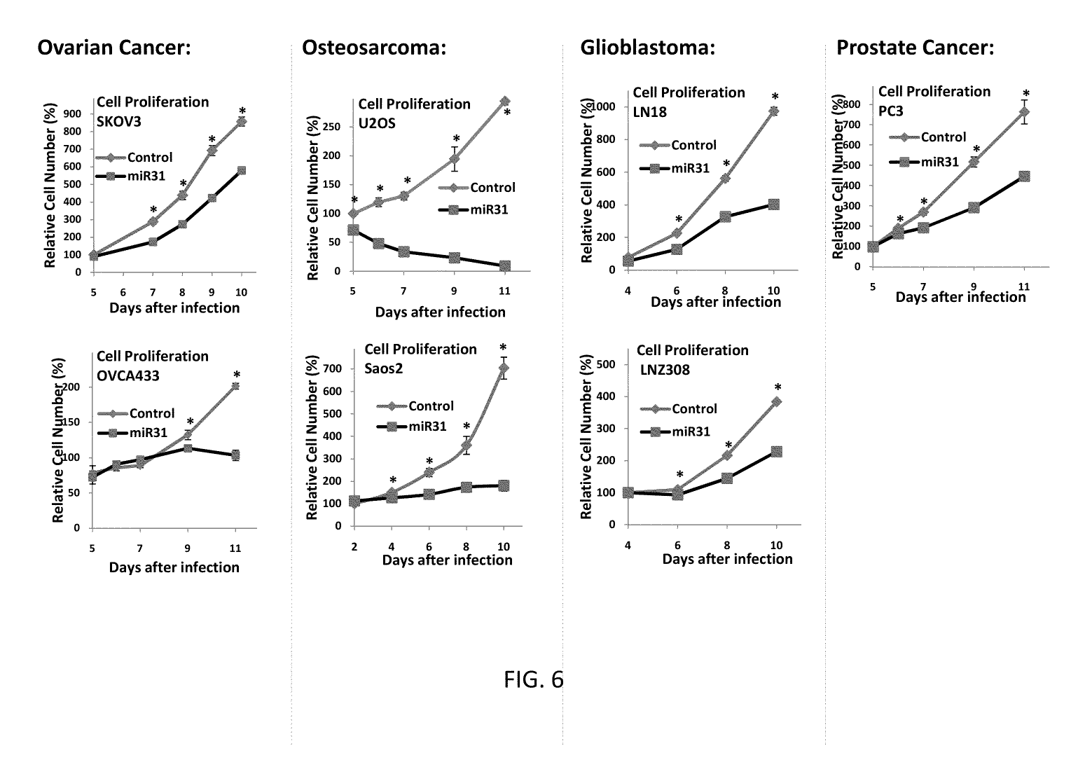

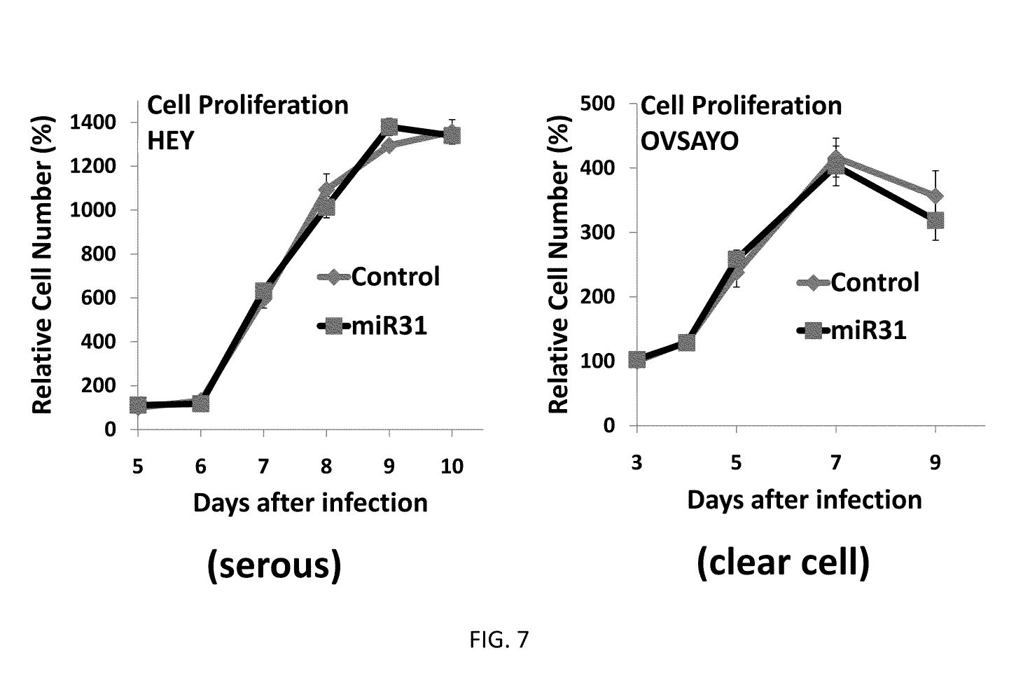

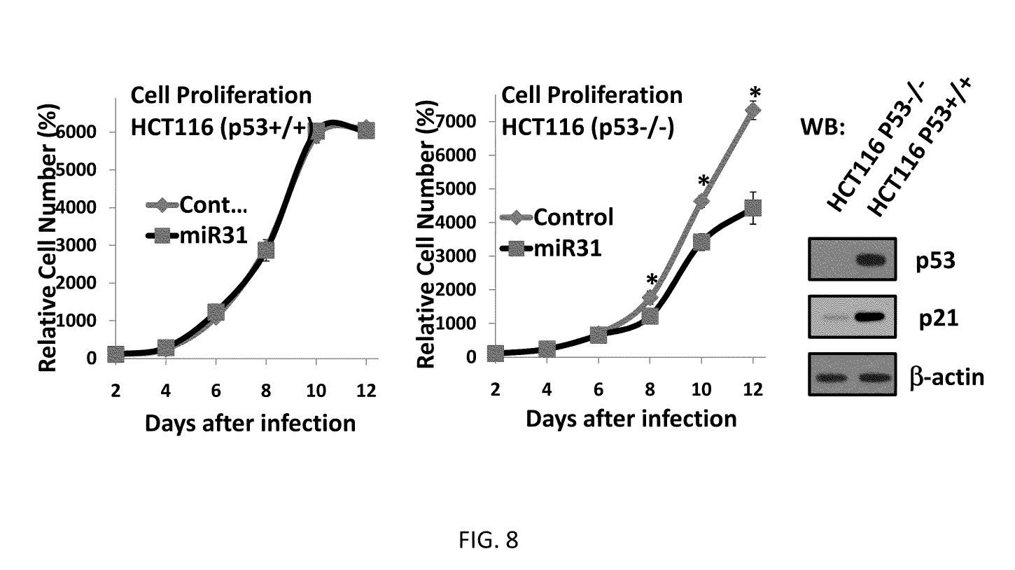

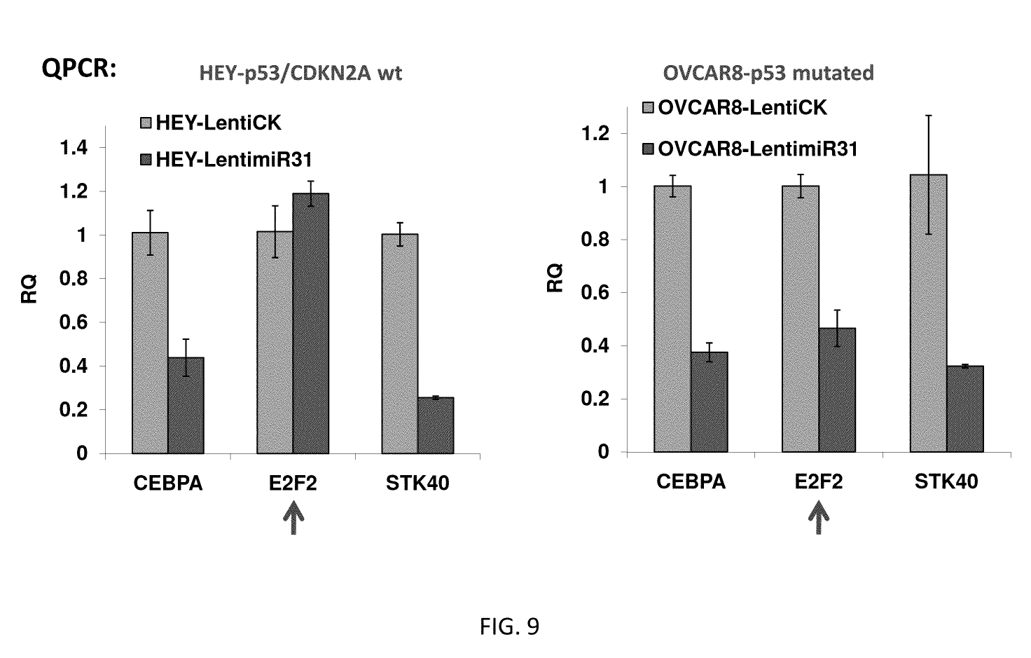

FIG. 6 shows the proliferation inhibitory effect of miR31 in certain cancer cell lines. FIG. 7 demonstrates that miR31 overexpression does not alter the proliferation of other ovarian cancer cell lines with wt p53/CDKN2A. FIG. 8 shows that the proliferation inhibitory effect of miR31 is associated with defects in the p53 pathway. FIG. 9 demonstrates analysis of some exemplary miR-31 targets.

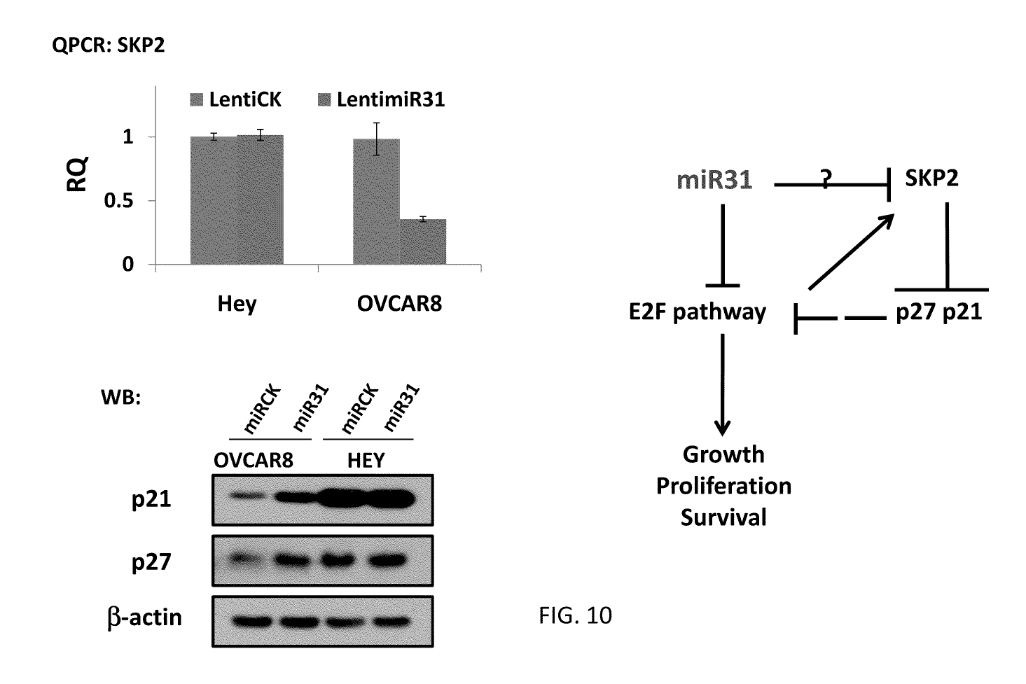

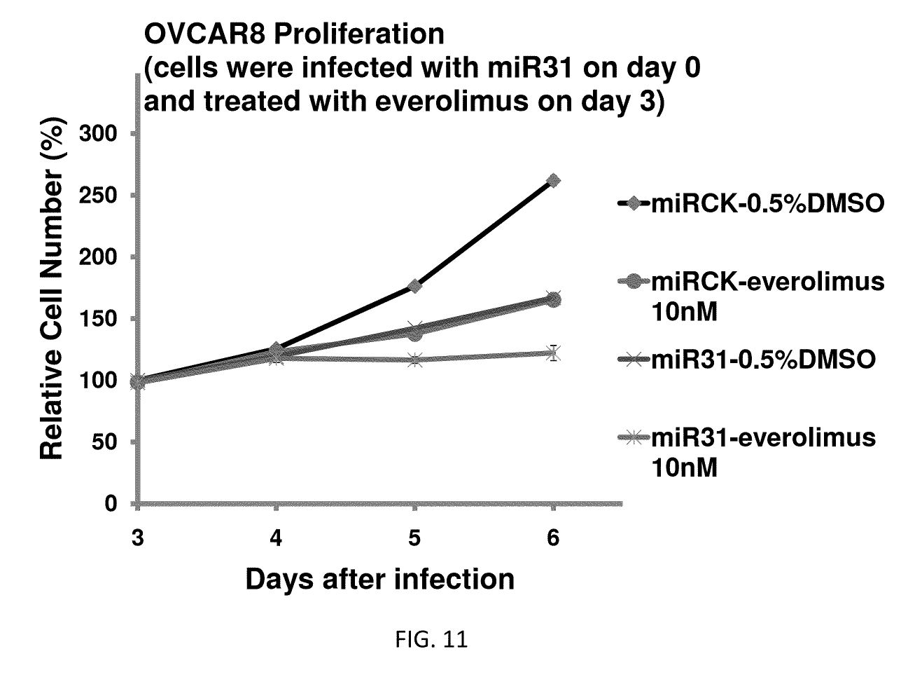

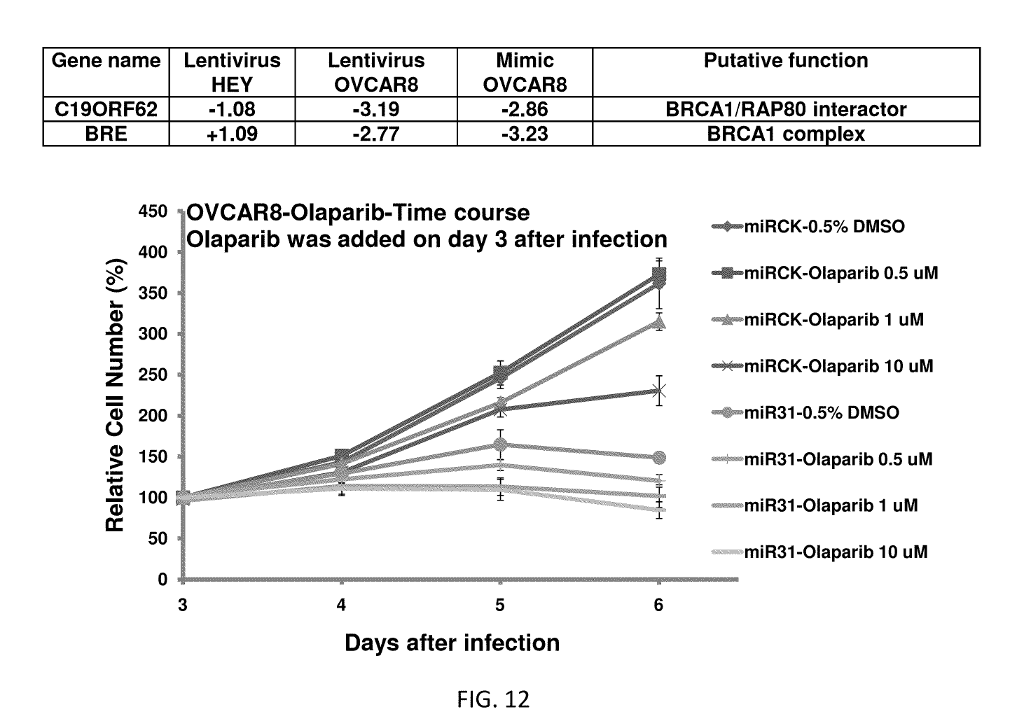

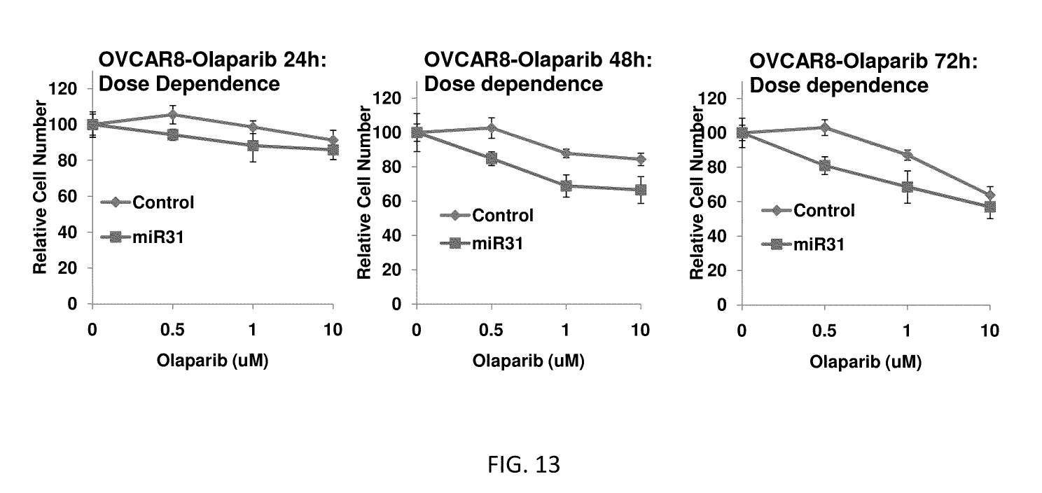

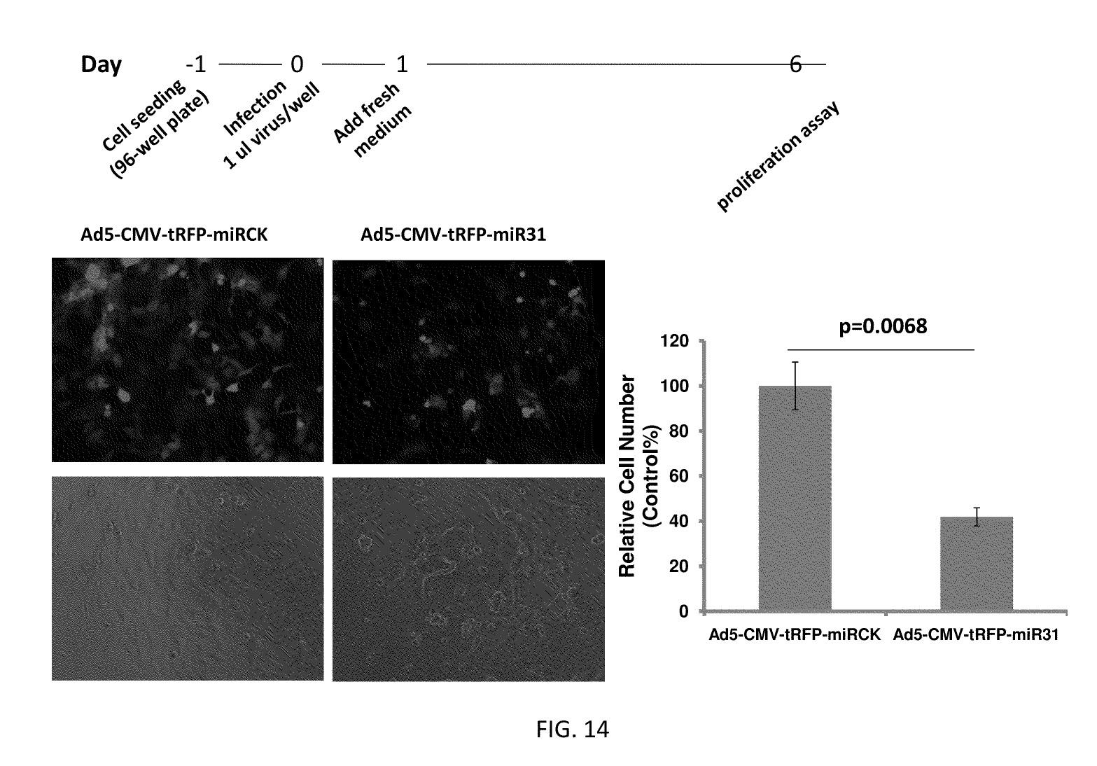

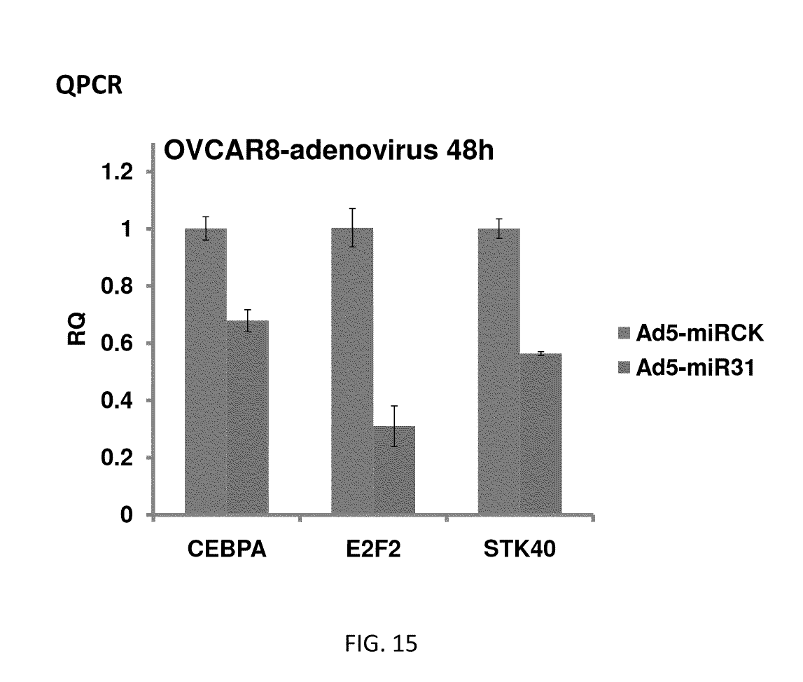

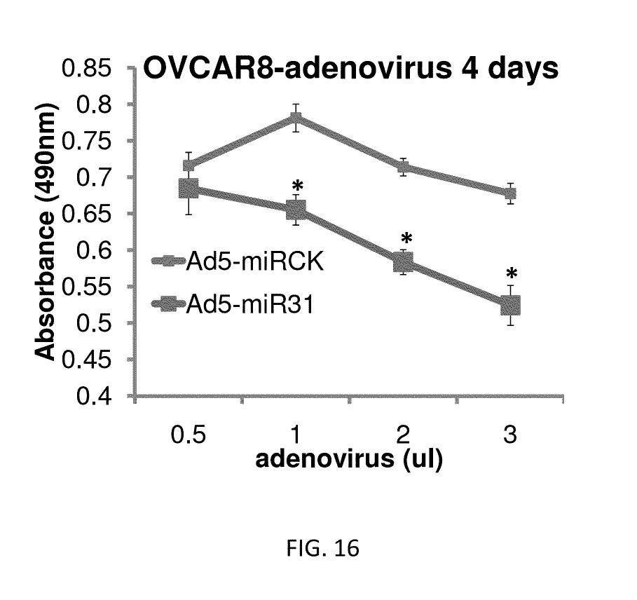

In certain embodiments of the invention, SKP2 is a target for the transcription factor E2F. In specific cases, SKP2 is a critical component of the SKP2-SCF complex, which acts as an E3 ligase to target p27, p21 and other substrates for ubiquitylation and degradation. In some embodiments, SKP2 has oncogenic activity. Overexpression of SKP2 is frequently observed in human cancers. FIG. 10 shows that SKP2 plays critical roles in the cell proliferation inhibitory function of miR31, in certain embodiments of the invention. FIG. 11 illustrates the additive effect of miR31 and everolimus on OVCAR8 cells. FIG. 12 demonstrates the additive effect of miR31 and Olaparib on OVCAR8 cells. FIG. 13 shows that miR31 sensitized OVCAR8 to Olaparib. FIG. 14 shows that adenoviral vector Ad5-CMV-tRFP-miR31 inhibited OVCAR8 cell proliferation. FIG. 15 demonstrates that MiR31 adenovirus downregulated miR31 targets CEBPA, E2F2 and STK40. FIG. 16 shows that miR31 adenovirus dose-dependently inhibited OVCAR8 cell proliferation.

### Example 5

### Significance of Certain Embodiments of the Invention

In this study, motivated by the hypothesis that regulatory defects play an early role in the molecular changes and progression of ovarian cancer, we profiled miRNAs and their target genes in serous epithelial ovarian cancer. For further study, we focused here on miR-31, which was underexpressed in both tumors and cell lines. By manipulating miR-31 in vitro, we were able to demonstrate widespread effects on gene expression, as had previously been indicated by the public target prediction databases. A number of the demonstrated miR-31 targets (e.g., E2F2) were differentially expressed between cancer and normal cells, and may represent genes involved in cancer initiation. Other miR-31 targets were potentially significant from the standpoint of cancer progression; specifically, miR-31 had widespread effects on genes correlated with poor prognosis in late stage serous epithelial ovarian cancer, making this miRNA an attractive therapeutic target.

Based on our results, miR-31 fits many of the features of a tumor suppressor in serous ovarian cancer, as it is both deleted and underexpressed, inhibits cancer cell proliferation, and increases caspase-mediated apoptosis. Recently, miR-31 was found by Valastyan et al. to inhibit metastasis in breast cancer (24). With our data demonstrating anti-proliferative effects of miR-31 in ovarian cancer, osteosarcoma, and prostate cancer, our study establishes an important role for miR-31 in multiple cancer types. Interestingly, however, miR-31 was not found to affect proliferation in vitro of MDA-MB-231 human breast cancer cells (24), and of the six demonstrated miR-31 targets in MDA-MB-231 in the Valastyan study, only two, M-RIP and RDX, were similarly downregulated (p<0.01) by miR-31 in our OVCAR-8 cells. This suggests that miR-31 may regulate different processes in different cancers, dependent on the cell of origin of the cancer.

Our gene signature of miR-31 overexpression might help to reveal specific miR-31-impacted genes that could contribute to the progression of the disease. For example, E2F transcription factor dimerization partner 2 (TFDP2), was repressed by miR-31 overexpression and individually correlated with poor prognosis in the Tothill data, and E2F2, while not correlated in the Tothill dataset with prognosis, was correlated with increasing grade and has been shown elsewhere to be correlated with poor prognosis in ovarian cancer in other patient cohorts (43). CEBPA, which is deregulated in various cancers, was correlated here with poor prognosis; more recently, altered expression of CEBPA in prostate cancer was linked to alterations in E2F complexes and the E2F-RB pathway (44).

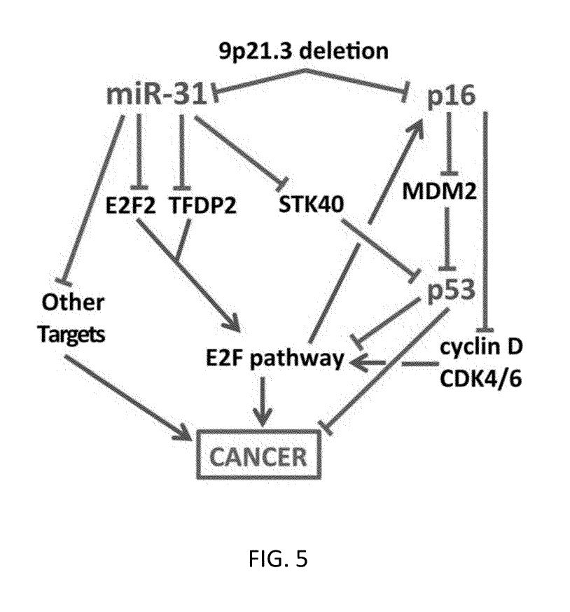

Mutations in TP53 are observed in approximately 10-20% of early cancers and up to 80% of advanced serous cancers, and correlate with metastatic potential (45, 46). The CDKN2A/ARF/p16 gene, which normally triggers p53 degradation, is located along with miR-31 in a region of common deletion at 9p21. Furthermore, studies have established a link between the RB/E2F pathway and the p53 response, where deregulated overexpression of E2Fs in quiescent cells normally leads via p16 to an induction of p53-dependent apoptosis (31, 32). Another miR-31 target, serine threonine kinase 40 (STK40), is a repressor of p53-mediated transcription (47). The associations drawn here between miR-31 and genes related to the p53 pathway suggests a model in which miR31 plays a tumor suppressor role in multiple cell types, acting alone and/or in concert with p16 and p53 to regulate the cell cycle and cancer (FIG. 5). Our analysis of multiple cell lines showed that those with inactive p53 pathway were vulnerable to miR-31 overexpression, while those with functional p53 were not, which further indicates a synergistic link between miR-31 deletion and p53 pathway inactivation.

Therapeutic delivery of miRNAs as a means of suppressing tumor-promoting genes has potential as a cancer treatment (48), though this approach has often been demonstrated for a particular miRNA using a single cell line or model system. Strategies to treat ovarian cancer could well include miR-31. At the same time, however, our study indicates that not all cancers would be susceptible to miR-31, such as those cancers which have a functional p53 pathway (including normal and intact CDKN2A/ARF/p16 gene) or which are not driven by E2F2. This scenario observed with miR-31 could apply to the use of miRNA therapeutics in general. In the case of established therapies that directly target specific genes, such as anti-HER2 Herceptin or anti-ER tamoxifen in breast cancer, only tumors that express specific molecular markers may respond. In the future, knowledge of the specific pathways targeted by a given miRNA, as well as which gene biomarkers predict therapeutic response to that miRNA, will likely be needed to properly assess the efficacy of miRNA therapeutics in controlling cancer in at least some patients.

### Example 6

### Exemplary Materials and Methods

### Cell Cultures

After obtaining informed consent from each study participant, primary cultures of normal ovarian surface epithelium (NOSE) were performed as previously described (49). The epithelial origin of cultured NOSE cells was confirmed using immunohistochemistry, and only cultures containing >90% epithelial cells were used. OVCA433, U2O5, and PC3 were kindly provided by Drs. J. Wolf, L. Donehower, and M. Ittmann, respectively. Cancer cell lines were cultured in DMEM (Invitrogen), RPMI 1640 (Invitrogen), McCoy's 5a modified medium (Invitrogen), or MCDB105/M199 (Sigma), with 10-20% heat-inactivated fetal bovine serum and 1% penicillin-streptomycin solution (Invitrogen) (see elsewhere herein for details).

### Gene Expression Profiling and Small RNA Sequencing

Total RNA was extracted from human NOSE cultures (n=9), serous ovarian cancer cell lines (n=7), and serous ovarian adenocarcinomas (n=17) using the mirVana miRNA Isolation Kit (ABI). RNA quality and the presence of small RNAs were inspected on a 2100 Bioanalyzer (Agilent). Gene expression profiles were generated on HumanWG-6 v3.0 BeadChips (Illumina) at Texas Children's Cancer Center Genomics and Proteomics Core Laboratory. Array datasets have been deposited into the Gene Expression Omnibus (GSE16709).

Small RNA library construction was performed using the DGE-Small RNA Sample Prep Kit (Illumina) (n=4 NOSE; n=4 cell lines, n=8 tumors). Purified cDNA was quantified with the Quant-iT PicoGreen dsDNA Kit (Invitrogen) and diluted to 3 pM for sequencing on the Illumina 1G Genome Analyzer (University of Houston). MicroRNA profiles were generated essentially as described in (50) and elsewhere herein.

Overexpression of miR-31

OVCAR-8 cells were transfected in 6-well plates (2.5×10⁵ cells/well) using 7.5 μl Lipofectamine 2000 Transfection Reagent (Invitrogen) and 3 μg hsa-mir-31 mimic (90 nM) (Dharmacon). Control groups were cells treated with transfection reagent alone (mock transfection), and cells transfected with miRIDIAN hairpin inhibitor negative control #1 or mimic negative control #2 (Dharmacon). Cells were harvested 48 hours after transfection, and gene expression profiling was performed as described above (n=3 for each inhibitor or mimic; n=3 for each negative control inhibitor or mimic).

For functional assays including proliferation and apoptosis measurements, CMV-TurboRFP-mir31-IRES-puroR(OpenBiosystem Cat. No. HMR4842-99855788) was packaged and used to infect the various cell lines, according to manufacturer's instructions. The DNA plasmid carrying non-targeting sequence (OpenBiosystem Cat. No. HMR4867) was used as negative control.

### Quantitative Real-Time PCR (QPCR)

QPCR validation of microarray data was performed on samples independent of those used in microarray experiments (n=5 for each transfection condition). Total RNA (500 ng) was reverse transcribed in a 50 μl reaction using 250 U Superscript III reverse transcriptase and random hexamers (Invitrogen). Custom primer sequences are provided in Supporting Methods. QPCR was performed on an ABI Prism 7500 Sequence Detection System using SYBR Green PCR Master Mix (ABI) in a 20 μl reaction and human β-actin (ACTB) as an endogenous control.

### Molecular Profile Analysis

Differentially expressed genes and miRNAs were identified using t-test on log-transformed data and fold change. All P-values were two-sided. Expression values were visualized as color maps using the Java TreeView software (51). MiRNAs predicted to target differentially expressed mRNAs were identified using TargetScanHuman (release 5.0) (52), PicTar (53), and miRanda (September 2008) (54). Retrieval of putative miRNA-mRNA pairs was facilitated by SigTerms software (55). Transcriptional targets of E2F2 were defined as the set of genes elevated according to dataset GSE7655 and bound by E2F2 according to ref (25). GSEA was executed using public software from the Broad Institute (see corresponding website). To score each of the Tothill et al. serous ovarian tumors (GSE9891) for similarity to our miR-31 gene signature, we derived a “t-score” for Tothill tumor in relation to the miR-31 signature as previously described (56, 57).

We obtained the CGH dataset from GEO (GSE12040). The 2464 BAC clones (each printed in triplicate) were first collapsed into the 644 cytoband loci represented by those genes. For each profile, the tumor: normal log ratios were averaged by cytoband. A fold change of 1.25 was used for defining gain or loss events within each cytoband. CGH data from the TCGA (see cancergenome website at the NIH) were from batch 9, and 11-13.

### Proliferation and Apoptosis Assays

Three or five days after infection, cells were washed once with PBS and trypsinized. Two thousand and five hundred miR-31 overexpressed or non-targeting control overexpressed cells were resuspended in 100 μl of culture medium and seeded onto 96-well plate. Cells were allowed to settle down for two hours followed by ATP quantitation-based Promega (Madison, Wis.) CellTiter-Glo cell proliferation assay (Cat. No. G7570) or MTS-based CellTiter 96 cell proliferation assay (Cat. No. G3580) for counting viable cells. Promega Caspase-Glo 3/7 Assay (Cat. No. G8090) was carried out according to the manufacturer's instructions as a sensitive assay for apoptosis.

### Primary Culture of Human Normal Ovarian Surface Epithelial Cells

Permission to perform all experiments was obtained from the Institutional Review Board for Baylor College of Medicine and its affiliated institutions (H-15188, H-22124, H-22119). After obtaining informed consent from each study participant, primary cultures of normal ovarian surface epithelium (NOSE) were performed as previously described (Kruk et al., 1990). Briefly, brushings of the ovarian surface were performed in pre- and postmenopausal women undergoing clinically indicated surgery. Women with evidence of endometriosis, cancer, or inflammatory conditions at the time of surgery were excluded from participation. Cells were cultured in 1:1 MCDB105/M199 (Sigma) with 10% heat-inactivated fetal bovine serum, 1% penicillin-streptomycin solution (Invitrogen), and 5 ng/ml epidermal growth factor (Invitrogen). The epithelial origin of cultured cells was confirmed using immunohistochemistry to examine the expression of vimentin and cytokeratins, and only cultures containing >90% epithelial cells were used.

### Cancer Cell Lines

HEY, OV-90, OVCAR-3, and SKOV3 were obtained from the American Type Culture Collection. OVCAR-8 and OVCAR-5 were obtained from the NCI-Frederick Cancer DCTD Tumor/Cell Line Repository. OVCA433, U2OS, and PC3 were kindly provided by Drs. J. Wolf, L. Donehower, and M. Ittmann, respectively. The OVCAR-8 and OVCAR-5 cells were cultured in RPMI 1640 (Invitrogen) with 10% heat-inactivated fetal bovine serum and 1% penicillin-streptomycin solution (Invitrogen). The OVCAR-3 cell line was cultured in RPMI 1640 (Invitrogen) with 20% heat-inactivated fetal bovine serum and 1% penicillin-streptomycin solution (Invitrogen). The HEY and OVCA433 cell lines were cultured in DMEM (Invitrogen) with 10% heat-inactivated fetal bovine serum and 1% penicillin-streptomycin solution (Invitrogen). SKOV3 cells were cultured in McCoy's 5a modified medium (Invitrogen) with 10% heat-inactivated fetal bovine serum and 1% penicillin-streptomycin solution (Invitrogen). The OV-90 cell line was cultured in a ratio of 1:1 MCDB105/M199 (Sigma) with 15% heat-inactivated fetal bovine serum and 1% penicillin-streptomycin solution (Invitrogen). U2OS was cultured in DMEM (Invitrogen) with 10% heat-inactivated fetal bovine serum and 1% penicillin-streptomycin solution (invitrogen). PC3 was cultured in RPMI 1640 (Invitrogen) with 20% heat-inactivated fetal bovine serum and 1% penicillin-streptomycin solution (Invitrogen).

### Gene Expression Profiling and Small RNA Sequencing

Total RNA was extracted from human NOSE cultures (n=9), serous ovarian cancer cell lines (n=7), and serous ovarian adenocarcinomas (n=17) using the mirVana miRNA Isolation Kit (ABI). RNA quality and the presence of small RNAs were inspected on a 2100 Bioanalyzer (Agilent). Gene expression profiles were generated on HumanWG-6 v3.0 BeadChips (Illumina) at Texas Children's Cancer Center Genomics and Proteomics Core Laboratory. The array platform consists of 48,803 transcript probes representing 24,384 unique named genes. Expression data were quantile normalized by applying a monotone linear spline to each array that mapped quantiles 0.01 to 0.99 in increments of 0.01 exactly to the corresponding quantiles of the standard using software kindly provided by Kerby Shedden (University of Michigan). All array datasets described in this study have been deposited into the Gene Expression Omnibus (GSE16709).

Small RNA library construction was performed using the DGE-Small RNA Sample Prep Kit (Illumina) (n=4 NOSE; n=4 cell lines, n=8 tumors). Purified cDNA was quantified with the Quant-iT PicoGreen dsDNA Kit (Invitrogen) and diluted to 3 pM for sequencing on the Illumina 1G Genome Analyzer (University of Houston). MicroRNA profiles were generated essentially as described in (Creighton et al., 2009). The raw sequence data were passed through a series of quality control filters to discard reads without a 3′ adapter, reads with copy number <4, reads that were <10 nucleotides, and reads with >10 consecutive identical nucleotides. The remaining usable sequence reads were aligned to human miRNA hairpin precursor sequences in miRBase (release 11.0; see Microrna Sanger website) (Griffiths-Jones et al., 2008), with up to 3 mismatches tolerated in an alignment. If a read aligned equally well to multiple precursors, its copy number was equally apportioned. For each mature miRNA sequence, we counted the number of reads that were exact matches, allowing a 4 nucleotide extension into the precursor sequence on each side of the mature miRNA. Each miRNA profile was scale normalized using the total number of usable sequence reads and then 40 units were added to each miRNA expression value.

Overexpression of miR-31

For profiling cells with overexpression of miR-31, transfection conditions for each ovarian cancer cell line were optimized for maximum efficiency in preliminary experiments using Dy547-labeled miRIDIAN hairpin inhibitor and mimic transfection controls (Dharmacon). Because the specificity of miRNA silencing is determined by the seed sequence, we examined the selected cell lines for aberrant expression of seed-matched family members that might hamper knockdown or overexpression efficiency, and simultaneously targeted those miRNAs. OVCAR-8 cells were transfected in 6-well plates (2.5×10⁵ cells/well) using 7.5 μl Lipofectamine 2000 Transfection Reagent (Invitrogen) and 3 μg hsa-mir-31 mimic (90 nM) (Dharmacon). Control groups were cells treated with transfection reagent alone (mock transfection), and cells transfected with miRIDIAN hairpin inhibitor negative control #1 or mimic negative control #2, which are based on C. elegans miRNAs that have minimal sequence identity to human miRNAs (Dharmacon). Cells were harvested 48 hours after transfection, and gene expression profiling was performed as described above (n=3 for each inhibitor or mimic; n=3 for each negative control inhibitor or mimic).

For functional assays including proliferation and apoptosis measurements, CMV-TurboRFP-mir31-IRES-puro^{R} (OpenBiosystem Cat. No. HMR4842-99855788) was packaged and used to infect the various cell lines. The DNA plasmid carrying non-targeting sequence (OpenBiosystem Cat. No. HMR4867) was used as negative control. Briefly, miR-31 or control plasmid, as well as the helper virus DNA plasmids carrying gag, pol, VSVG and tat were transfected into 293T cells by Lipofectamine 2000 (Invitrogen) according to manufacturer's instructions. Forty-eight hours post transfection, cell medium containing virus were syringe filtered (0.45 μm) and added onto various cell lines with appropriated dilution in the presence of polybrene (8 μg/ml). The virus was changed with fresh cell medium 6 hours post infection. The infection efficiency was measured by examining the cells under fluorescence microscope and was determined to be >95%.

### Quantitative Real-Time PCR (QPCR)

QPCR validation of microarray data was performed on samples independent of those used in microarray experiments (n=5 for each transfection condition). Whereas our microarray experiments compared gene expression in cells treated with miRNA inhibitor or mimic relative to negative control, our QPCR experiments measured differences in these groups compared to mock transfected cells. This allowed us to examine for unexpected effects after treatment with negative control mimic, which has the seed sequence UGUACUA, predicted to target 92 human genes (TargetScan).

Total RNA (500 ng) was reverse transcribed in a 50 μl reaction using 250 U Superscript III reverse transcriptase and random hexamers (Invitrogen). cDNA was diluted 50-fold and 5 μl was used for each QPCR reaction. Custom primers were designed using Primer Express software (ABI) and primer sequences are as follows: CEPBA forward, AAGAAGTCGGTGGACAAGAACAG (SEQ ID NO:1); CEBPA reverse, GCAGGCGGTCATTGTCACT (SEQ ID NO:2); STK40 forward, CGTGCACAGAGACCTGAAGCT (SEQ ID NO:3); STK40 reverse, GAGGCAGAAGTTGGTGATGGTT (SEQ ID NO:4); E2F2 forward, TGAGCTTCAAGCACCTGACTGA (SEQ ID NO:5); E2F2 reverse, TTGCCAACAGCACGGATATC (SEQ ID NO:6). QPCR was performed on an ABI Prism 7500 Sequence Detection System using SYBR Green PCR Master Mix (ABI) in a 20 μl reaction and human β-actin (ACTB) as an endogenous control. Reaction conditions were 2 min at 50° C., 10 min at 95° C., 40 cycles of 15 sec at 95° C. (denaturation) and 1 min at 60° C. (annealing/extension). Each sample was analyzed in triplicate and a nontemplate control (nuclease-free water) was included for each primer set. The relative quantity (RQ) of transcript was calculated using the 2^{-ΔΔC}_{T} method with the average ΔC_{T} of the mock transfection group as the calibrator (Livak and Schmittgen, 2001). P-values were two-sided.

### Expression Data Analysis

Differentially expressed genes and miRNAs were identified using t-test on log-transformed data and fold change. All P-values were two-sided. Fold changes (ratio of group averages) were computed essentially as in (Creighton et al., 2008). Expression values were visualized as color maps using the Java TreeView software (Saldanha, 2004). MiRNAs predicted to target differentially expressed mRNAs were identified using TargetScanHuman (release 5.0; see TargetScan website) (Friedman et al., 2009), PicTar (see PicTar website) (Krek et al., 2005), and miRanda (September 2008; see Microrna website) (Betel et al., 2008). For mRNAs that were overexpressed in cancer, we searched for targeting miRNAs that were underexpressed, and vice-versa. Retrieval of putative miRNA-mRNA pairs was facilitated by SigTerms software (see SigTerms website) (Creighton et al., 2008). Transcriptional targets of E2F2 were defined as the set of genes elevated according to dataset GSE7655 and bound by E2F2 according to ref (Georlette et al., 2007).

MiR-31-induced gene expression patterns were evaluated using GSEA. GSEA was executed using public software from the Broad Institute (see website) with the following settings: “classic” enrichment statistic; permutation by gene set with 1000 permutations; and “ratio of classes” used for ranking genes. For GSEA, the entire set of genes represented in the given functional array dataset were ranked by ratio of experimental/control. When more than one array probe corresponded to a given gene, the probe with the greatest variation between experimental and control groups was used to represent the gene.

To score each of the Tothill et al. serous ovarian tumors (using expression profile dataset GSE9891) for similarity to our miR-31 gene signature, we derived a “t-score” for Tothill tumor in relation to the miR-31 signature in the same manner to that in previous studies (Creighton et al., 2008; Gibbons et al., 2009). Briefly, the t-score was defined as the two-sided statistic comparing the average of the miR-31-induced genes with that of the repressed genes within each tumor. The gene expression values in the Tothill dataset were first normalized to standard deviations from the median before computing the t-score. The mapping of transcripts or genes between the two array datasets was made on the Entrez Gene identifier; where multiple human array probe sets referenced the same gene, the probe set with the highest variation represented the gene. Progression-free survival was capped at 5 years.

### DNA Copy Number Analysis

We obtained the CGH dataset from GEO (GSE12040). The 2464 BAC clones (each printed in triplicate) were first collapsed into the 644 cytoband loci represented by those genes. By integrating information across neighboring genes, binning provides a useful balance between minimizing noise and maximizing mapping resolution (Castro et al., 2009; Lapoinet et al., 2007). For each profile, the tumor: normal log ratios were averaged by cytoband. A fold change of 1.25 was used for defining gain or loss events within each cytoband, comparable to what has been used elsewhere (Castro et al., 2009; Lapointe et al., 2007) (and which helps compensate for array data compression and normal tissue contamination in the tumor). CGH data from the TCGA (see NIH Cancer Genome website) were from batch 9, and 11-13.

### Proliferation and Apoptosis Assays

Three or five days after infection, cells were washed once with PBS and trypsinized. Two thousand and five hundred miR-31 overexpressed or non-targeting control overexpressed cells were resuspended in 100 μl of culture medium and seeded onto 96-well plate. Cells were allowed to settle down for two hours followed by ATP quantitation-based Promega (Madison, Wis.) CellTiter-Glo cell proliferation assay (Cat. No. G7570) or MTS-based CellTiter 96 cell proliferation assay (Cat. No. G3580) for counting viable cells. The assay was performed every one or two days, as indicated, to generate the in vitro proliferative kinetics. Promega Caspase-Glo 3/7 Assay (Cat. No. G8090) was carried out according to the manufacturer's instructions as a sensitive assay for apoptosis.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

### REFERENCES

1. Auersperg, N., Wong, A., Choi, K., Kang, S., & Leung, P. (2001) Endocr Rev 22, 255-288.

2. Jemal, A., Siegel, R., Ward, E., Hao, Y., Xu, J., Murray, T., & Thun, M. (2008) CA Cancer J Clin 58, 71-96.

3. Cho, K. & Shih, I. (2009) Annu Rev Pathol 4, 287-313.

4. Seidman, J., Horkayne-Szakaly, I., Haiba, M., Boice, C., Kurman, R., & Ronnett, B. (2004) Int J Gynecol Pathol 23, 41-44.

5. Levanon, K., Crum, C., & Drapkin, R. (2008) J Clin Oncol 26, 5284-5293.

6. Bell, D. (2005) Mod Pathol 18 Suppl 2, S19-32.

7. Bonome, T., Lee, J., Park, D., Radonovich, M., Pise-Masison, C., Brady, J., Gardner, G., Hao, K., Wong, W., Barrett, J., et al. (2005) Cancer Res 65, 10602-10612.

8. Wu, L. & Belasco, J. (2008) Mol Cell 29, 1-7.

9. Corney, D. & Nikitin, A. (2008) Histol Histopathol 23, 1161-1169.

10. Deng, S., Cann, G., Croce, C., Coukos, G., & Zhang, L. (2008) Cell Cycle 7, 2643-2646.

11. Wijnhoven, B., Michael, M., & Watson, D. (2007) Br J Surg 94, 23-30.

12. Zhao, H., Wang, D., Du, W., Gu, D., & Yang, R. (2009) Crit Rev Oncol Hematol E-pub June 9.

13. Yu, S., Chen, H., Yang, P., & Chen, J. (2007) DNA Cell Biol 26, 283-292.

14. Iorio, M., Visone, R., Di Leva, G., Donati, V., Petrocca, F., Casalini, P., Taccioli, C., Volinia, S., Liu, C., Alder, H., et al. (2007) Cancer Res 67, 8699-8707.

15. Nam, E., Yoon, H., Kim, S., Kim, H., Kim, Y., Kim, J., Kim, J., & Kim, S. (2008) Clin Cancer Res 14, 2690-2695.

16. Zhang, L., Volinia, S., Bonome, T., Cann, G., Greshock, J., Yang, N., Liu, C., Giannakakis, A., Alexiou, P., Hasegawa, K., et al. (2008) Proc Natl Acad Sci USA 105, 7004-7009.

17. Bearfoot, J., Choong, D., Gorringe, K., & Campbell, I. (2008) Clin Cancer Res 14, 7246-7250.

18. Dahiya, N., Sherman-Baust, C., Wang, T., Davidson, B., Shih, I., Zhang, Y., Wood, W. r., Becker, K., & Morin, P. (2008) PLoS One 3, e2436.

19. Wyman, S., Parkin, R., Mitchell, P., Fritz, B., O'Briant, K., Godwin, A., Urban, N., Drescher, C., Knudsen, B., & Tewari, M. (2009) PLoS ONE 4, e5311.

20. Yang, N., Kaur, S., Volinia, S., Greshock, J., Lassus, H., Hasegawa, K., Liang, S., Leminen, A., Deng, S., Smith, L., et al. (2008) Cancer Res 68, 10307-10314.

21. Sorrentino, A., Liu, C., Addario, A., Peschle, C., Scambia, G., & Ferlini, C. (2008) Gynecol Oncol 111, 478-486.

22. Pollack, J., Sørlie, T., Perou, C., Rees, C., Jeffrey, S., Lonning, P., Tibshirani, R., Botstein, D., Børresen-Dale, A., & Brown, P. (2002) Proc Natl Acad Sci USA 99, 12963-12968.

23. Nowee, M., Snijders, A., Rockx, D., de Wit, R., Kosma, V., Hämäläinen, K., Schouten, J., Verheijen, R., van Diest, P., Albertson, D., et al. (2007) J Pathol 213, 46-55.

24. Valastyan, S., Reinhardt, F., Benaich, N., Calogrias, D., Szász, A., Wang, Z., Brock, J., Richardson, A., & Weinberg, R. (2009) Cell 137, 1032-1046.

25. Georlette, D., Ahn, S., MacAlpine, D., Cheung, E., Lewis, P., Beall, E., Bell, S., Speed, T., Manak, J., & Botchan, M. (2007) Genes Dev 21, 2880-2896.

26. Cayirlioglu, P., Ward, W., Silver Key, S., & Duronio, R. (2003) Mol Cell Biol 23, 2123-2134.

27. Tothill, R., Tinker, A., George, J., Brown, R., Fox, S., Lade, S., Johnson, D., Trivett, M., Etemadmoghadam, D., Locandro, B., et al. (2008) Clin Cancer Res. 14, 5198-5208.

28. Sasaki, S., Kitagawa, Y., Sekido, Y., Minna, J., Kuwano, H., Yokota, J., & Kohno, T. (2003) Oncogene 22, 3792-3798.

29. Haupt, Y., Maya, R., Kazaz, A., & Oren, M. (1997) Nature 387, 296-299.

30. Zhang, Y. & Xiong, Y. (1999) Mol Cell 3, 579-591.

31. Weinberg, R. (2007) in The biology of cancer, ed. Weinberg, R. (Garland Science, Taylor & Francis Group, LLC, New York), pp. 307-356.

32. Sherr, C. & McCormick, F. (2002) Cancer Cell 2, 103-112.

33. Wolf, J., Mills, G., Bazzet, L., Bast, R. J., Roth, J., & Gershenson, D. (1999) Gynecol Oncol. 75, 261-266.

34. Samouelian, V., Maugard, C., Jolicoeur, M., Bertrand, R., Arcand, S., Tonin, P., Provencher, D., & Mes-Masson, A. (2004) Cancer Chemother Pharmacol 54, 497-504.

35. Itamochi, H., Kigawa, J., Kanamori, Y., Oishi, T., Bartholomeusz, C., Nahta, R., Esteva, F., Sneige, N., Terakawa, N., & Ueno, N. (2007) Mol Cancer Ther 6, 227-235.

36. Havrilesky, L., Alvarez, A., Whitaker, R., Marks, J., & Berchuck, A. (2001) Gynecol Oncol. 83, 491-500.

37. Kubo, A., Nakagawa, K., Varma, R., Conrad, N., Cheng, J., Lee, W., Testa, J., Johnson, B., Kaye, F., & Kelley, M. (1999) Clin Cancer Res 5, 4279-4286.

38. Mitsuuchi, Y., Johnson, S., Selvakumaran, M., Williams, S., Hamilton, T., & Testa, J. (2000) Cancer Res 60, 5390-5394.

39. Yaginuma, Y. & Westphal, H. (1992) Cancer Res 52, 4196-4199.

40. Plasencia, C., Dayam, R., Wang, Q., Pinski, J., Burke, T. J., Quinn, D., & Neamati, N. (2005) Mol Cancer Ther 4, 1105-1113.

41. Grossel, M., Baker, G., & Hinds, P. (1999) J Biol Chem 274, 29960-29967.

42. Lanzi, C., Cassinelli, G., Cuccuru, G., Supino, R., Zuco, V., Ferlini, C., Scambia, G., & Zunino, F. (2001) Prostate 48, 254-264.

43. Reimer, D., Sadr, S., Wiedemair, A., Stadlmann, S., Concin, N., Hofstetter, G., Müller-Holzner, E., Marth, C., & Zeimet, A. (2007) Clin Cancer Res 13, 144-151.

44. Yin, H., Lowery, M., & Glass, J. (2009) Prostate 69, 1001-1016.

45. Berchuck, A., Kohler, M., Marks, J., Wiseman, R., Boyd, J., & Bast, R. J. (1994) Am J Obstet Gynecol 170, 246-252.

46. Havrilesky, L., Darcy, M., Hamdan, H., Priore, R., Leon, J., Bell, J., Berchuck, A., & Gynecologic_Oncology_Group_Study (2003) J Clin Oncol 21, 3814-3825.

47. Huang, J., Teng, L., Liu, T., Li, L., Chen, D., Li, F., Xu, L., Zhai, Z., & Shu, H. (2003) Biochem Biophys Res Commun 309, 774-778.

48. Kota, J., Chivukula, R., O'Donnell, K., Wentzel, E., Montgomery, C., Hwang, H., Chang, T., Vivekanandan, P., Torbenson, M., Clark, K., et al. (2009) Cell 137, 1005-1017.

49. Kruk, P. A., Maines-Bandiera, S. L., & Auersperg, N. (1990) Lab Invest 63, 132-136.

50. Creighton, C., Reid, J., & Gunaratne, P. (2009) Brief Bioinform E-pub March 30.

51. Saldanha, A. J. (2004) Bioinformatics 20, 3246-3248.

52. Friedman, R. C., Farh, K. K., Burge, C. B., & Bartel, D. P. (2009) Genome Res 19, 92-105.

53. Krek, A., Grun, D., Poy, M. N., Wolf, R., Rosenberg, L., Epstein, E. J., MacMenamin, P., da Piedade, I., Gunsalus, K. C., Stoffel, M., et al. (2005) Nat Genet 37, 495-500.

54. Betel, D., Wilson, M., Gabow, A., Marks, D. S., & Sander, C. (2008) Nucleic Acids Res 36, D149-153.

55. Creighton, C. J., Nagaraja, A. K., Hanash, S. M., Matzuk, M. M., & Gunaratne, P. H. (2008) RNA 14, 2290-2296.

56. Creighton, C., Casa, A., Lazard, Z., Huang, S., Tsimelzon, A., Hilsenbeck, S., Osborne, C., & Lee, A. (2008) J Clin Oncol 26, 4078-4085.

57. Gibbons, D., Lin, W., Creighton, C., Zheng, S., Berel, D., Yang, Y., Raso, M., Liu, D., Wistuba, I., Lozano, G., et al. (2009) PLoS ONE 4, e5401.

Means G D, Kilgore M W, Mahendroo M S, Mendelson C R, Simpson E R. Mol Endocrinol. 1991 December; 5(12):2005-13.

Kruk, P. A., Maines-Bandiera, S. L., & Auersperg, N. (1990) Lab Invest 63, 132-136.

Creighton, C., Reid, J., & Gunaratne, P. (2009) Brief Bioinform E-pub March 30.

Griffiths-Jones, S., Saini, H. K., van Dongen, S., & Enright, A. J. (2008) Nucleic Acids Res 36, D154-158.

Livak, K. J. & Schmittgen, T. D. (2001) Methods 25, 402-408.

Creighton, C., Casa, A., Lazard, Z., Huang, S., Tsimelzon, A., Hilsenbeck, S., Osborne, C., & Lee, A. (2008) J Clin Oncol 26, 4078-4085.

Saldanha, A. J. (2004) Bioinformatics 20, 3246-3248.

Friedman, R. C., Farh, K. K., Burge, C. B., & Bartel, D. P. (2009) Genome Res 19, 92-105.

Krek, A., Grun, D., Poy, M. N., Wolf, R., Rosenberg, L., Epstein, E. J., MacMenamin, P., da Piedade, I., Gunsalus, K. C., Stoffel, M., et al. (2005) Nat Genet 37, 495-500.

Betel, D., Wilson, M., Gabow, A., Marks, D. S., & Sander, C. (2008) Nucleic Acids Res 36, D149-153.

Creighton, C. J., Nagaraja, A. K., Hanash, S. M., Matzuk, M. M., & Gunaratne, P. H. (2008) RNA 14, 2290-2296.

Georlette, D., Ahn, S., MacAlpine, D., Cheung, E., Lewis, P., Beall, E., Bell, S., Speed, T., Manak, J., & Botchan, M. (2007) Genes Dev 21, 2880-2896.

Gibbons, D., Lin, W., Creighton, C., Zheng, S., Berel, D., Yang, Y., Raso, M., Liu, D., Wistuba, I., Lozano, G., et al. (2009) PLoS ONE 4, e5401.

Castro, P., Creighton, C., Ozen, M., Berel, D., Mims, M., & Ittmann, M. (2009) Neoplasia 11, 305-312.

Lapointe, J., Li, C., Giacomini, C., Salari, K., Huang, S., Wang, P., Ferrari, M., Hernandez-Boussard, T., Brooks, J., & Pollack, J. (2007) Cancer Res 67, 8504-8510.

Certain innovative aspects of the invention are defined in more detail in the appending claims. Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of treating cancer in an individual, wherein the cancer is ovarian, urothelial, osteosarcoma, glioblastoma, or prostate, comprising the step of delivering a therapeutically effective amount of microRNA-31 to the individual.

2. The method of claim 1, wherein the cancer is deficient in p53 signaling.

3. The method of claim 1, wherein the microRNA-31 is delivered to the individual orally, intraperitoneally, or intravenously.

4. The method of claim 1, wherein the microRNA-31 is delivered in a viral vector.

5. The method of claim 4, wherein the viral vector is a lentiviral vector, adenoviral vector, adeno-associated vector, or retroviral vector.

6. The method of claim 1, wherein the microRNA-31 targets E2F2, TFDP2, MDM2, or STK40.

7. The method of claim 1, further comprising the step of delivering an additional anti-cancer therapy to the individual.

8. The method of claim 7, wherein the additional anti-cancer therapy is surgery, chemotherapy, radiation, hormone therapy, immunotherapy, or a combination thereof.


SEQUENCE LISTING (9 sequences)

SEQ ID NO: 1 - DNA, 23 bp, Artificial Sequence
  Synthetic primer
aagaagtcggtggacaagaacag

SEQ ID NO: 2 - DNA, 19 bp, Artificial Sequence
  Synthetic primer
gcaggcggtcattgtcact

SEQ ID NO: 3 - DNA, 21 bp, Artificial Sequence
  Synthetic primer
cgtgcacagagacctgaagct

SEQ ID NO: 4 - DNA, 22 bp, Artificial Sequence
  Synthetic primer
gaggcagaagttggtgatggtt

SEQ ID NO: 5 - DNA, 22 bp, Artificial Sequence
  Synthetic primer
tgagcttcaagcacctgactga

SEQ ID NO: 6 - DNA, 20 bp, Artificial Sequence
  Synthetic primer
ttgccaacagcacggatatc

SEQ ID NO: 7 - RNA, 71 nt, Artificial Sequence
  Synthetic primer
ggagaggaggcaagaugcuggcauagcuguugaacugggaaccugcuaugccaacauauugccaucuuucc

SEQ ID NO: 8 - RNA, 21 nt, Artificial Sequence
  Synthetic primer
aggcaagaugcuggcauagcu

SEQ ID NO: 9 - RNA, 22 nt, Artificial Sequence
  Synthetic primer
ugcuaugccaacauauugccau